(19) United States
(12) Patent Application Publication
(10) Pub. No.: US 2025/0389720 A1
(43) Pub. Date: Dec. 25, 2025

(54) Assay for Early Detection of Nasopharyngeal Carcinoma

(71) Applicant: University of Pittsburgh - Of the Commonwealth System of Higher Education, Pittsburgh, PA (US)

(72) Inventors: Kathy Ho Yen Shair, Sewickley, PA (US); Jian-Min Yuan, Pittsburgh, PA (US)

(21) Appl. No.: 18/860,952

(22) PCT Filed: Apr. 28, 2023
(86) PCT No.: PCT/US2023/066374

§ 371 Date: Oct. 28, 2024

Related U.S. Application Data
(60) Provisional application No. 63/336,590, filed on Apr. 29, 2022.

(51) Int. Cl. G01N 33/574; G01N 33/569; G01N 33/58

(52) U.S. Cl. CPC G01N 33/57407; G01N 33/56983; G01N 33/57484; G01N 33/582; G01N 2333/05; G01N 2469/20

(57) ABSTRACT

Early Nasopharyngeal Carcinoma (NPC) detection methods, devices, and kits are provided utilizing novel Epstein-Barr Virus (EBV) EBNA1 antigens to detect serum anti-EBNA1 IgA antibodies with superior correlation to NPC development within four years.

### CROSS REFERENCE TO RELATED APPLICATIONS

This application is the United States national phase of International Patent Application No. PCT/US23/66374 filed Apr. 28, 2023, and claims priority to U.S. Provisional Patent Application No. 63/336,590 filed Apr. 29, 2022, the disclosures of which are hereby incorporated by reference in their entireties.

The Sequence Listing associated with this application is filed in electronic format via Patent Center and is hereby incorporated by reference into the specification in its entirety. The name of the XML file containing the Sequence Listing is 2300839.xml. The size of the XML file is 7,541 bytes and the XML file was created on Apr. 27, 2023.

Nasopharyngeal carcinoma (NPC) is a leading head and neck cancer in Southeast Asia, particularly in southern China where NPC is endemic. Historically, NPC incidence in the 1980s was high in Chinese men in Hong Kong (30/100,000) and Singapore (19/100,000), moderate in Shanghai, China (5/100,000), and low in white men in the United States (0.5/100,000). Although NPC incidence has decreased over time, there were an estimated 133,000 new cases and 80,000 deaths from NPC worldwide in 2020. Early-stage NPC (I and II) is often asymptomatic, and therefore most NPC cases are diagnosed later (stage III and IV). Five-year overall survival decreases from 90% when diagnosed at stage I to 58% at stage IV. Although environmental exposures and genetic factors may contribute to the risk of NPC, more than 97% of tumors are associated with latent Epstein-Barr virus (EBV). Additionally, elevated antibodies to EBV lytic proteins are considered a harbinger of NPC. Thus, a survey of EBV serology could yield crucial information to predict NPC risk and possibly provide target markers for vaccine development and efficacy evaluation.

Several EBV biomarkers have been proposed for NPC screening in high-risk populations. Plasma cell-free EBV DNA, which may reflect the release of EBV from apoptotic and/or necrotic cells in a tumor, can detect early-stage NPC. This improved detection at stage I and II is based on the levels, methylation pattern, and fragment size of cell-free EBV DNA. IgA antibodies against EBV viral capsid antigen p18 (VCA p18) and nuclear antigen 1 (EBNA1) have been evaluated for early detection of NPC in several high-risk populations. A two-step enzyme-linked immunosorbent assay (ELISA) approach to detect IgA against VCA p18 and EBNA1 followed by IgA against EBV early antigen nuclear protein extracts can achieve accurate early detection (sensitivity 96.7%, specificity 98%) in an Indonesian NPC-endemic cohort. Many of these previous studies were conducted in high-risk populations with cross-sectional design or short duration of follow-up. Therefore, the results only indicated whether NPC was present. Additional biomarkers may be required to assess NPC risk. Coghill et al. conducted a comprehensive serological survey for EBV biomarkers using a peptide array and found that a composite score of 14 EBV antibodies, including IgA against VCA p18 and EBNA1, had an estimated 85% sensitivity and 61% specificity in a general Taiwanese population cohort to determine NPC status on average 4.2 years before clinical diagnosis (Coghill A E, et al. Identification of a Novel, EBV-Based Antibody Risk Stratification Signature for Early Detection of Nasopharyngeal Carcinoma in Taiwan. Clinical Cancer Research 2018; 24(6):1305-14). Thirteen of these biomarkers were validated by surveying sera taken from individuals at the time of NPC diagnosis using an independent multiplex assay based on bacterial-expressed EBV proteins conjugated to beads (Simon J, Liu Z, Brenner N, Yu KJ, Hsu W-L, Wang C-P, et al. Validation of an Epstein-Barr Virus Antibody Risk Stratification Signature for Nasopharyngeal Carcinoma by Use of Multiplex Serology. Journal of Clinical Microbiology 2020; 58(5)). Given the low incidence of NPC in the general population, the next step is to improve upon the specificity of an NPC risk score to warrant implementation as a screening test.

### STATEMENT REGARDING FEDERAL FUNDING

This invention was made with government support under CA186873 and CA182876 awarded by the National Institutes of Health. The government has certain rights in the invention.

### SUMMARY

Provided herein are improved methods, devices, and kits for use in NPC screening and treatment.

According to an aspect of the invention, a device is provided. The device comprises a protein-binding substrate; and bound to the substrate, an isolated EBV EBNA1 protein produced in a mammalian cell that binds serum IgA of an NPC patient, the protein comprising an EBV EBNA1 protein with at least 50% of the GA region deleted.

The following clauses outline additional aspects, embodiments, and/or examples of the present invention.

Clause 1: A device comprising: a protein-binding substrate; and bound to the substrate, an isolated EBV EBNA1 protein produced in a mammalian cell that binds serum IgA of an NPC patient, the protein comprising an EBV EBNA1 protein with at least 50% of the GA region deleted.

Clause 2: The device of clause 1, wherein the EBV EBNA1 protein comprising a sequence of SEQ ID NO: 1 in which at least 120 contiguous amino acids between amino acids 89 and 328 are deleted.

Clause 3: The device of clause 1, wherein the EBV EBNA1 protein comprising a sequence of SEQ ID NO: 2 in which at least 95 contiguous amino acids between amino acids 91 and 284 are deleted.

Clause 4: The device of clause 1, wherein the EBV EBNA1 protein comprising a sequence of SEQ ID NO: 1 or SEQ ID NO: 2 in which at least 75% of the GA region is deleted.

Clause 5: The device of clause 1, wherein the EBV EBNA1 protein comprising a sequence of SEQ ID NO: 1 or SEQ ID NO: 2 in which at least 90% of the GA region is deleted.

Clause 6: The device of clause 1, wherein the EBV EBNA1 protein comprising a sequence of SEQ ID NO: 1 or SEQ ID NO: 2 in which the GA region is deleted.

Clause 7: The device of any one of clauses 1-6, wherein the EBV EBNA1 protein comprising a sequence of SEQ ID NO: 1, comprising one, two, or three of the amino acid substitutions E411D, H418L, and A439T.

Clause 8: The device of claim 1, wherein the EBV EBNA1 protein comprises at least an immunodominant epitope of SEQ ID NO: 2.

Clause 9: The device of any one of clauses 1-8, wherein the EBV EBNA1 protein has a higher amino acid sequence identity to an EBNA1 present in a nasopharyngeal carcinoma than SEQ ID NO: 1.

Clause 10: The device of clause 1, wherein the EBV EBNA1 protein comprises an amino acid sequence having at least 95%, at least 96%, at least 97%, at least 98%, or at least 99% sequence identity with SEQ ID NO: 2.

Clause 11: The device of clause 1, wherein the modified EBV EBNA1 protein has the amino acid sequence of SEQ ID NO: 4.

Clause 12: The device of any one of clauses 1-11, wherein the substrate is a wettable membrane.

Clause 13: The device of clause 12, wherein the wettable membrane is a nitrocellulose, nylon, or PVDF (polyvinylidene difluoride) membrane.

Clause 14: The device of any one of clauses 1-13, wherein the substrate is a wettable membrane and the modified EBV EBNA1 protein is bound to the membrane at two or more discrete locations, such as a slot blot or a dot blot.

Clause 15: The device of clause 14, further comprising positive and negative control proteins deposited at one or more discrete locations on the membrane.

Clause 16: The device of clause 15, comprising one or more positive control proteins deposited at one or more discrete locations on the membrane, wherein the positive control protein is human IgA.

Clause 17: A kit, comprising a device of any one of clauses 1-16, contained within packaging.

Clause 18: The kit of clause 17, further comprising anti-human-IgA primary antibody, optionally contained within a vessel.

Clause 19: The kit of clause 18, wherein the anti-human IgA primary antibody is labeled with a fluorescent moiety, an enzyme for activating a colorimetric or chemiluminescent substrate, or a radiolabel.

Clause 20: The kit of clause 17, further comprising a labeled secondary antibody that binds specifically to the anti-human IgA primary antibody.

Clause 21: The kit of clause 20, wherein the label is an enzyme for activating a colorimetric or chemiluminescent substrate.

Clause 22: The kit of clause 21, wherein the label is horseradish peroxidase, and the kit further comprises a colorimetric or chemiluminescent substrate of the horseradish peroxidase.

Clause 23: The kit of any one of clauses 17-22, wherein the substrate is a bead for use in flow cytometry, and the label is fluorescent.

Clause 24: The kit of clause 23, comprising two or more beads, packaged separately or together, with different fluorescent profiles.

Clause 25: The kit of clause 23, wherein the two or more beads are packaged in a container for processing together, and each of the two or more beads have different proteins or amounts of the modified EBV EBNA1 protein bound thereto.

Clause 26: The kit of clause 23, wherein the two or more beads are packaged separately in two or more containers for processing separately, and at least one of the two or more beads have the modified EBV EBNA1 protein bound thereto, and optionally two or more of the beads have different amounts of the modified EBV EBNA1 protein bound thereto.

Clause 27: The kit of any one of clauses 17-26, further comprising one or more reagents for identification of free EBV DNA in a patient's blood or plasma (e.g., cell-free plasma).

Clause 28: A method of treating a patient, comprising monitoring the patient for risk of developing NPC by determining if the patient has serum anti-EBV EBNA1 IgA antibodies by determining if serum IgA antibodies of the patient bind to an EBV EBNA1 protein produced in a mammalian cell that binds serum IgA of an NPC patient, the protein comprising an EBV EBNA1 protein with at least 50% of the GA region deleted using a device of any one of clauses 1-16, and when the patient has serum IgA antibodies that bind to the EBV EBNA1 protein, monitoring the patient for development of NPC, and when present, treating the patient for NPC

Clause 29: The method of clause 28, wherein the determining if the patient has serum anti-EBV EBNA1 IgA antibodies, comprises contacting the patient's serum with the device of any one of claims 1-9, and determining if IgA of the patient is bound to the EBV EBNA1 protein bound to the substrate of the device.

Clause 30: The method of clause 28 or 29, wherein when anti-EBNA1 IgA is detected, the patient is monitored regularly, such as one or more times within one two, three, or four years, e.g., every one, two, three, four, six, or 12 months, after detection of the anti-EBNA1 IgA in the patient.

Clause 31: The method of any one of clauses 28-30, wherein the IgA is detected by binding to a denaturing blot comprising the EBNA1 polypeptide, optionally of the EBV Akata strain.

Clause 32: The method of any one of clauses 28-31, further comprising identifying the presence of free EBV DNA in the patient's blood or plasma (e.g., cell-free plasma).

Clause 33: The method of any one of clauses 28-32, comprising treating the patient with an antiviral agent or EBV vaccine, such as mRNA-1189, EBV gp_350 Ferritin vaccine, or a pentavalent Epstein-Barr Virus-Like Particle vaccine.

Clause 34: A method of determining risk of development of NPC in a patient, comprising: determining if the patient has serum anti-EBV EBNA1 IgA antibodies by determining if serum IgA antibodies of the patient bind to an EBV EBNA1 protein produced in a mammalian cell that binds serum IgA of an NPC patient, the protein comprising an EBV EBNA1 protein with at least 50% of the GA region deleted using a device of any one of clauses 1-16.

### BRIEF DESCRIPTION OF THE DRAWINGS

FIG. 1A provides the amino acid sequence of a wild-type EBV EBNA1 (GenBank: CAA24816.1, B95-8, SEQ ID NO: 1). FIG. 1B. Amino acid sequences for EBNA1 Akata wild-type (EBNA1_Ak (SEQ ID NO: 2)), EBNA1 with the complete glycine/alanine (Gly/Ala) repeat removed (EBNA1dGGA_Ak (SEQ ID NO: 3)), and the Akata EBNA1 variant used in the serology screen library (EBNA1_Library_Ak (SEQ ID NO: 4)) are shown with glycine/alanine repeat amino acids highlighted (gray); polymorphisms in the immunodominant epitope region; *=stop codon.

FIGS. 2A-2C. Amino acid sequence of the EBNA1 probe used for risk assessment of nasopharyngeal carcinoma. (FIG. 2A) Comparison of the EBNA1 sequence and domains encoded by the EBV Akata strain used in the library screen (EBNA1_Library_Ak) compared to the full-length Genbank sequence (EBNA1_Ak) and the derivative construct with complete deletion of the Gly/Ala repeats (EBNA1dGGA_Ak). IDE denotes two immunodominant epitope regions, the Gly/Ala repeats denote a region with variable iterative repeats which naturally vary by EBV isolate and EBV strain. NLS, nuclear localization signal; DBD, DNA binding domain; DD1 and DD2, dimerization domains 1 and 2. Removal of the Gly/Ala repeats improves mammalian expression. (FIG. 2B) Comparison of the number of glycine and alanine residues in EBNA1 depicting differences in the EBV B958 strain, sequences identified from NPC biopsies, and the EBNA1_Library_Ak expression construct devoid of the majority of Gly/Ala repeats. (FIG. 2C) Amino acid sequence alignment of EBNA1 expression variants. Highlighted sequence denotes the Gly/Ala repeats. The sequences flanking the Gly/Ala repeat are identical (*) to the wild-type EBNA1 protein.

FIG. 3A depicts schematically an EBNA1 protein on a binding membrane. FIG. 3B depicts schematically a slot blot membrane comprising multiple deposits of an EBNA1 protein, and control locations. FIG. 3C provides a schematic of an exemplary method of protein analyte immunodetection.

FIG. 4. Overview of method used for serologic screening of EBV antibodies from the serum of individuals who later developed nasopharyngeal carcinoma compared to cohort matched controls that had no evidence of cancer. A new EBV mammalian expression library encoding 86 (Akata strain) proteins was used to survey antibodies against EBV proteins. IgA antibody against EBV nuclear antigen 1 (EBNA1) was the leading biomarker for discriminating incident NPC cases from controls.

FIG. 5 Number of IgG and IgA EBV proteins other than EBNA1 in 20 NPC case-control pairs in the Singapore Chinese Health Study (SCHS) discovery group. The number of EBV proteins (nEBV) that were detected by IgG_{H} or IgA_{H} (heavy chain-specific, cutoff=0) in NPC cases (black triangle) were compared to control samples (grey inverted triangle). EBNA1 detection is excluded. EBV proteins are classified as lytic, latent or all gene classes (including unassigned genes) and organized by time to NPC diagnosis (dx) in years. Each point represents one sample and n=number of case-control pairs for each time interval. NPC cases diagnosed with NPC within 2 years in the <3 group are highlighted (blue triangle). Median values (red bar) are indicated. Statistical significance is labeled as *p<0.05, ** p<0.01, *** p<0.001, calculated by a Mann-Whitney test.

FIG. 6. Study population and design. The Singapore Chinese Health Study (SCHS) and Shanghai Cohort Study (SCS) represent NPC endemic and non-endemic (moderate-risk) populations, respectively. Pre-diagnostic sera were identified for use based on time to NPC diagnosis (median, range in years) and sufficient serum availability, and were divided into discovery and validation groups. Each NPC case was matched with a healthy control based on pre-defined criteria in the methods. Case-control pairs were surveyed against the entire 86 EBV ORF expression library for IgA, IgG, and IgM, or tested for EBNA1 IgA only.

FIG. 7 EBV proteins detected by IgA in the SCHS discovery group excluding EBNA1. EBV proteins that were detected by IgA in sera from incident NPC cases (18 proteins), controls (1 protein), or both (7 proteins) are shown. Proteins in blue were unique to incident NPCs within 4 years of NPC diagnosis. Proteins that discriminated cases from controls are bolded and denoted as *p<0.05, ** p<0.01.

FIG. 8 shows Mikrogen recomLine EBV IgA commercial assay has a lower sensitivity than the multiplex immunoblot assay (SCHS+SCS combined data) using the Akata EBNA1 protein, as described herein.

FIG. 9. Graphical representation of EBNA1 constructs from Table 4.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. As used herein, embodiments “comprising” one or more stated elements or steps also include, but are not limited to embodiments “consisting essentially of” and consisting of these stated elements or steps.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” or “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). As used herein, the terms “treating”, or “treatment” refer to a beneficial or desired result, such as improving one of more functions, or symptoms of a disease.

Unless stated otherwise, nucleotide sequences are recited herein in a 5′ to 3′ direction, and amino acid sequences are recited herein in an N-terminal to C-terminal direction according to convention.

As used herein, the “treatment” or “treating” of a patient means administration to a patient by any suitable dosage regimen, procedure and/or administration route of a composition, device, or structure with the object of achieving a desirable clinical/medical end-point, including but not limited to, any suitable treatment for NPC, and also includes monitoring the patient for development of NPC tumors by any useful method. A patient may monitored for development of stage I or II NPC, and may be treated when the NPC is stage I or stage II. An amount of any reagent or therapeutic agent, administered by any suitable route, effective to treat a patient is an amount capable of including but not limited to, prior to discovery of NPC in a patient antiviral drugs or other therapies for treatment or prevention of EBV, such as prophylactic drugs or vaccines, or, where NPC is detected after routine screening, any suitable treatment for NPC, such as administration of cisplatin or 5-fluorouracil in effective amounts once NPC is detected, immunotherapies, cytokine therapies, radiotherapies, or any other therapy useful for treatment of NPC. A therapeutic agent may be administered by any effective route. A therapeutic agent may be administered as a single dose, at regular or irregular intervals, in amounts and intervals as dictated by any clinical parameter of a patient or graft organ or tissue, or continuously.

In further detail, a number of preventative or therapeutic measures may be implemented on a positive test for serological anti-EBNA1 IgA as described herein. Clinical follow-up may be implemented, including MRI and endoscopy (see, e.g., King AD. MR Imaging of Nasopharyngeal Carcinoma. Magn Reson Imaging Clin N Am. 2022 February; 30(1):19-33). In addition to the present disclosure, other early detection tests may be used to monitor a patient for development of NPC, such as a cell-free EBV DNA test (e.g., the Take2 Prophecy™ Test for Nasopharyngeal Cancer; an EBV serology ELISA test for both EBNA1 and VCA IgA (see, Ji MF, et al. Incidence and mortality of nasopharyngeal carcinoma: interim analysis of a cluster randomized controlled screening trial (PRO-NPC-001) in southern China. Ann Oncol. 2019 Oct. 1; 30(10):1630-1637 and ClinicalTrials.gov Identifier: NCT00941538)); and ELISA test for both EBNA1 and VCA IgA with BNLF2b total immunoglobulin (ClinicalTrials.gov Identifier: NCT04085900). An NPC EBNA1 inhibitor, e.g., VK-2019 (Hau PM, et al. Targeting Epstein-Barr Virus in Nasopharyngeal Carcinoma. Front Oncol. 2020 May 14; 10:600. 2020 May 14; 10:600). A number of EBV vaccines are also in development, such as mRNA-1189 (Moderna, lipid nanoparticle with four EBV mRNAs, including envelope glycoproteins gp42, gp220, gH and gL) an EBV gp_350 Ferritin Vaccination (see, e.g., Kanekiyo M, et al. Rational Design of an Epstein-Barr Virus Vaccine Targeting the Receptor-Binding Site. Cell. 2015 Aug. 27; 162(5):1090-100, also, ClinicalTrials.gov Identifiers: NCT04645147 and NCT05683834); and a pentavalent Epstein-Barr Virus-Like Particle Vaccine (Escalante GM, et al. A Pentavalent Epstein-Barr Virus-Like Particle Vaccine Elicits High Titers of Neutralizing Antibodies against Epstein-Barr Virus Infection in Immunized Rabbits. Vaccines (Basel). 2020 Apr. 6; 8(2):169).

The term “contacting” refers to placement in direct physical association; includes both in solid and liquid form. “Contacting” is often used interchangeably with “exposed.” In some cases, “contacting” includes transfecting, such as transfecting a nucleic acid molecule into a cell. In other examples, “contacting” refers to incubating a molecule (such as an antibody) with a biological sample.

An “isolated” or “purified” biological component (such as a nucleic acid, peptide, protein, protein complex, or particle) refers to a component that has been substantially separated, produced apart from, or purified away from other components in a preparation or other biological components in the cell of the organism in which the component occurs, that is, other chromosomal and extrachromosomal DNA and RNA, and proteins. Nucleic acids, peptides and proteins that have been “isolated” or “purified”, thus, include, for example and without limitation, nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. A preparation may be purified such that the biological component represents at least 50%, such as at least 70%, at least 90%, at least 95%, or greater, of the total biological component content of the preparation.

A nucleic acid molecule (a nucleic acid) refers to a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” The term includes single- and double-stranded forms of DNA. A polynucleotide may include either or both naturally occurring and modified nucleotides linked together by naturally occurring and/or non-naturally occurring nucleotide linkages.

A first nucleic acid is said to be operably linked to a second nucleic acid when the first nucleic acid is placed in a functional relationship with the second nucleic acid. Generally, operably linked DNA sequences are contiguous (e.g., in cis) and, where the sequences act to join two protein coding regions, in the same reading frame (e.g., open reading frame or ORF), for example to produce a fusion protein. Operably linked nucleic acids include a first nucleic acid contiguous with the 5′ or 3′ end of a second nucleic acid. In other examples, a second nucleic acid is operably linked to a first nucleic acid when it is embedded within the first nucleic acid, for example, where the nucleic acid construct includes (in order) a portion of the first nucleic acid, the second nucleic acid, and the remainder of the first nucleic acid.

A “codon-optimized” nucleic acid refers to a nucleic acid sequence that has been altered such that the codons are optimal for expression in a particular system (such as a particular species of group of species). For example, a nucleic acid sequence can be optimized for expression in yeast cells. Codon optimization does not alter the amino acid sequence of the encoded protein.

A conservative substitution is a substitution of one amino acid residue in a protein sequence for a different amino acid residue having similar biochemical properties. Typically, conservative substitutions have little to no impact on the activity of a resulting polypeptide. For example, a TPD polypeptide sequence may include one or more conservative substitutions (for example 1-10, 2-5, or 10-20, or no more than 2, 5, 10, 20, 30, 40, or 50 substitutions) yet retains the antigenic structure and function of the wild-type protein, namely, in the context of the present disclosure, the ability to bind to a nanobody co-expressed in a yeast cell. A polypeptide can be produced to contain one or more conservative substitutions by manipulating the nucleotide sequence that encodes that polypeptide using, for example, standard procedures such as site-directed mutagenesis or PCR. Methods are provided herein to ascertain proper expression of any TPD sequence.

A polypeptide is a polymer in which the monomers are amino acid residues which are joined together through amide bonds. When the amino acids are alpha-amino acids, either the L-optical isomer or the D-optical isomer can be used. The terms “polypeptide”, “peptide”, or “protein” as used herein are intended to encompass any amino acid sequence and include proteins and modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Conservative amino acid substitutions are those substitutions that, when made, least or minimally interfere with the properties of the original protein, that is, in the context of the end-use, the structure and function of the protein is conserved and not significantly changed by such substitutions, and may be identified by use of matrices, such as the BLOSUM series of matrices, and other matrices. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl, or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

As used herein, the term “epitope” refers to a physical structure or moiety on a molecule that interacts with an antibody or antibody fragment. In terms of proteins or polypeptides, the primary amino acid sequence can define an epitope, but secondary and tertiary protein structure, as well as post-translational modifications, can define an epitope, though secondary and tertiary structure typically follows from the primary amino acid sequence. For example, the dGGA-EBNA1 proteins for use in the immunodetection methods, devices, and kits described herein are produced in mammalian cells, such as HEK293 cells, to produce a protein with mammalian post-translational modifications. Portions of a natural protein can contain an epitope present in the complete natural protein and typically react to antibodies raised to the natural protein. In the case of the immunodominant epitope (IDE) of EBNA1, which is a small portion of the total protein, a polypeptide containing portions of the natural protein containing that IDE, and produced in a mammalian cell, such as an HEK293 cell, are expected to bind to serum IgA antibodies specific to the same IDE present in the native, e.g. wild-type protein.

A recombinant nucleic acid refers to a nucleic acid molecule (or protein or virus) that is not naturally occurring or has a sequence that is made by an artificial combination of two otherwise separated segments of sequence. This artificial combination is accomplished by chemical synthesis or, more commonly, by the artificial manipulation of isolated segments of nucleic acids. The term recombinant includes nucleic acids and proteins that have been altered solely by addition, substitution, or deletion of a portion of a natural nucleic acid molecule or protein.

“Sequence identity” refers to the similarity between nucleic acid or amino acid sequences is expressed in terms of the similarity between the sequences, otherwise referred to as sequence identity. Sequence identity may be measured in terms of percentage identity (or similarity or homology); the higher the percentage, the more similar the two sequences are. Homologs, orthologs, or variants of a polypeptide will possess a relatively high degree of sequence identity when aligned using standard methods. Methods of alignment of sequences for comparison are well-known in the art. Various programs and alignment algorithms are described in the art (see, e.g., Chao J, et al. Developments in Algorithms for Sequence Alignment: A Review. Biomolecules. 2022 Apr. 6; 12(4):546. doi: 10.3390/biom12040546).

Once aligned, the number of matches may be determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide are typically characterized by possession of at least about 75%, for example, at least about 80%, 85%, 90%, 91%, 92%, 93%, 94%, 95%, 96%, 97%, 98%, or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example and without limitation, by the local homology algorithm of Smith & Waterman, by the homology alignment algorithm of Needleman & Wunsch, by the search for similarity method of Pearson & Lipman, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. One example of a useful algorithm is PILEUP. PILEUP uses a simplification of the progressive alignment method of Feng & Doolittle. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package (see, e.g., Chao J, et al. Biomolecules. 2022 Apr. 6; 12(4):546).

Another example of algorithms that is suitable for determining percent sequence identity and sequence similarity is the BLAST algorithm. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix. An oligonucleotide is a linear polynucleotide sequence of up to about 100 nucleotide bases in length (see, e.g., Chao J, et al. Biomolecules. 2022 Apr. 6; 12(4):546).

As used herein, reference to “at least 80% identity” (or similar language) refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Complementary refers to the ability of polynucleotides (nucleic acids) to hybridize to one another, forming inter-strand base pairs. Base pairs are formed by hydrogen bonding between nucleotide units in polynucleotide strands that are typically in antiparallel orientation. Complementary polynucleotide strands can base pair (hybridize) in the Watson-Crick manner (e.g., A to T, A to U, C to G), or in any other manner that allows for the formation of duplexes. In RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by the nucleobase content of the strands, the presence of mismatches, the length of complementary sequences, salt concentration, temperature, with the melting temperature (Tm) lowering with shorter complementary sequences, increased mismatches, and increased stringency. Perfectly matched sequences are said to be “fully complementary”, though one sequence (e.g., a target sequence in an mRNA) may be longer than the other.

A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

By “expression” or “gene expression,” it is meant the overall flow of information from a gene or functional/structural RNA, and a polyadenylation sequence), to produce a gene product (typically a protein, optionally post-translationally modified or a functional/structural RNA). A “gene” refers to a functional genetic unit for producing a gene product, such as RNA or a protein in a cell, or other expression system encoded on a nucleic acid and comprising: a transcriptional control sequence, such as a promoter and other cis-acting elements, such as transcriptional response elements (TREs) and/or enhancers; an expressed sequence that may encode a protein (referred to as an open-reading frame or ORF), and a polyadenylation sequence. By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent—for example an amount of the respective inducer is available to the expression system (e.g., cell), or factors causing suppression of a gene are unavailable or displaced—effective to cause expression of the gene.

Unless otherwise explained, all technical and scientific terms used herein have the same meaning as commonly understood by one of ordinary skill in the art to which this disclosure belongs. It is to be understood that all base sizes or amino acid sizes, and all molecular weight or molecular mass values, given for nucleic acids or polypeptides are approximate, and are provided for description. Unless otherwise indicated, polymer molecular weight is expressed as number-average molecular weight (Mn). Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Nucleic acids and vectors encoding the described fusion proteins may be provided. In some non-limiting examples, disclosed is a recombinant vector, such as a yeast plasmid, that expresses the disclosed fusion proteins. One of skill in the art can readily use the genetic code to construct a variety of functionally equivalent nucleic acids, such as nucleic acids which differ in sequence but which encode the same protein sequence due to codon degeneracy. In some embodiments, the polynucleotide is codon-optimized for expression in mammalian cells.

Exemplary nucleic acids may be prepared by cloning techniques, e.g., as are broadly-known and implemented either commercially, or in the art. Multiple textbooks and reference manuals describe and provide examples of useful and appropriate cloning and sequencing techniques, and instructions sufficient to direct persons of skill through such techniques are known. Commercial and public product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), Addgene, and Applied Biosystems (Foster City, Calif.), as well as many other commercial sources.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Reagents described herein for use in detecting the presence of anti-EBNA1 IgA antibodies in a patient's blood include antibodies and dGGA-EBNA1 protein, as described in further detail below. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any molecule, such as a human IgA or an EBV EBNA1 protein. Natural antibodies comprise two heavy chains and two light chains and are bi-valent. The interaction between the variable regions of heavy and light chain forms a binding site capable of specifically binding an antigen (e.g., a paratope). The term “V_{H}” refers to a heavy chain variable region of an antibody. The term “V_{L}” refers to a light chain variable region of an antibody. Antibodies may be derived from natural sources, or partly or wholly synthetically produced. Many antibodies and fragments thereof are available from commercial sources. An antibody may be monoclonal or polyclonal. The antibody may be a member of any immunoglobulin class, including any of the human classes: IgG, IgM, IgA, IgD, and IgE.

One reagent used in the methods, devices, and kits described herein is an anti-human IgA antibody that is labeled with a fluorophore or enzyme. Labeled anti-human IgA antibodies may be monoclonal or polyclonal and may be prepared in any suitable species. Labeled anti-human IgA antibodies are broadly-available, such as Cy™3 AffiniPure Goat anti-Human Serum IgA, α chain specific (AB_2337721, Jackson ImmunoResearch), Alexa Fluor® 488 anti-IgA antibody (rabbit monoclonal, Abcam), Goat anti-Human IgA (Heavy chain) Secondary Antibody, HRP (ThermoFisher), Goat anti-Human IgA-HRP (SouthernBiotech), among others. Where the anti-human IgA antibody is not labeled, a labeled antibody that binds the anti-human IgA antibody may be used to detect binding. Secondary antibodies, such as HRP-conjugated secondary antibodies for binding to the anti-human IgA antibodies are broadly-available commercially from a large number of sources.

The term “antibody fragment” refers to any derivative of an antibody which is less than full-length. In exemplary embodiments, the antibody fragment retains at least a significant portion of the full-length antibody's specific binding ability. Examples of antibody fragments, but are not limited to, Fab, Fab′, F(ab′)2, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. The antibody fragment may be produced by any means. For instance, the antibody fragment may be enzymatically or chemically produced by fragmentation of an intact antibody, or it may be recombinantly or synthetically produced. The antibody fragment may optionally be a single chain antibody fragment. Alternatively, the fragment may comprise multiple chains which are linked together, for instance, by disulfide linkages. The fragment may also optionally be a multi-molecular complex. A functional antibody fragment may consist of at least about 50 amino acids or at least about 200 amino acids. Antibody fragments also include miniaturized antibodies or other engineered binding reagents, such as scFvs, that exploit the modular nature of antibody structure, comprising, often as a single chain, one or more antigen-binding or epitope-binding (e.g., paratope) sequences and, at a minimum, any other amino acid sequences needed to ensure appropriate specificity, delivery, and stability of the composition.

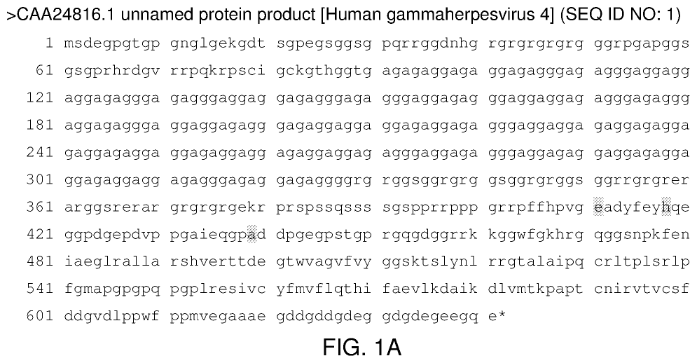

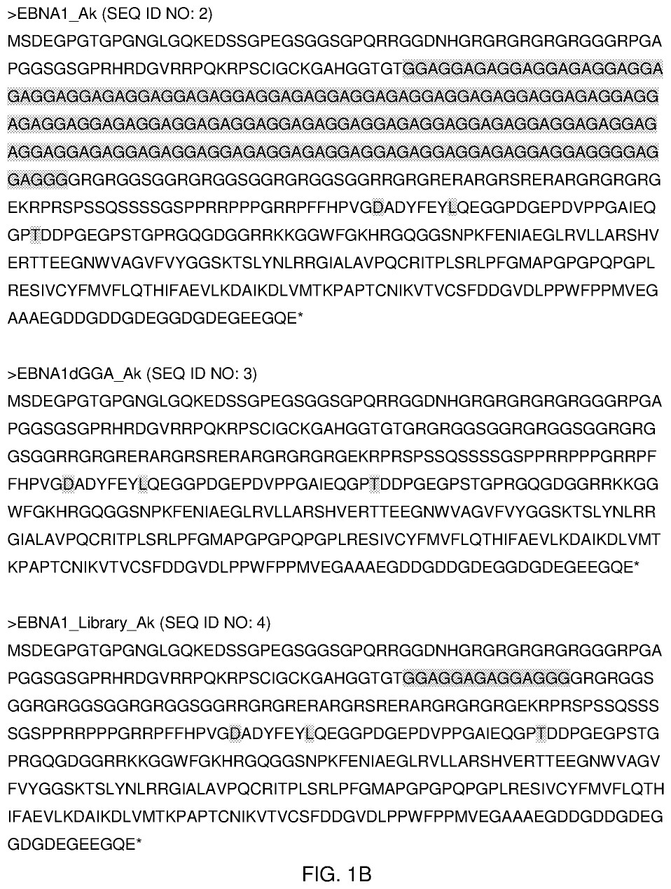

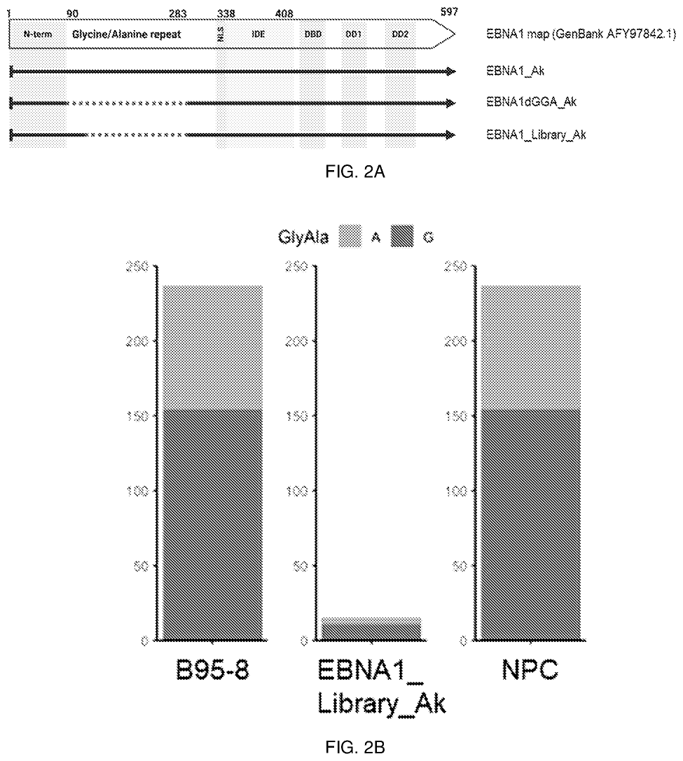

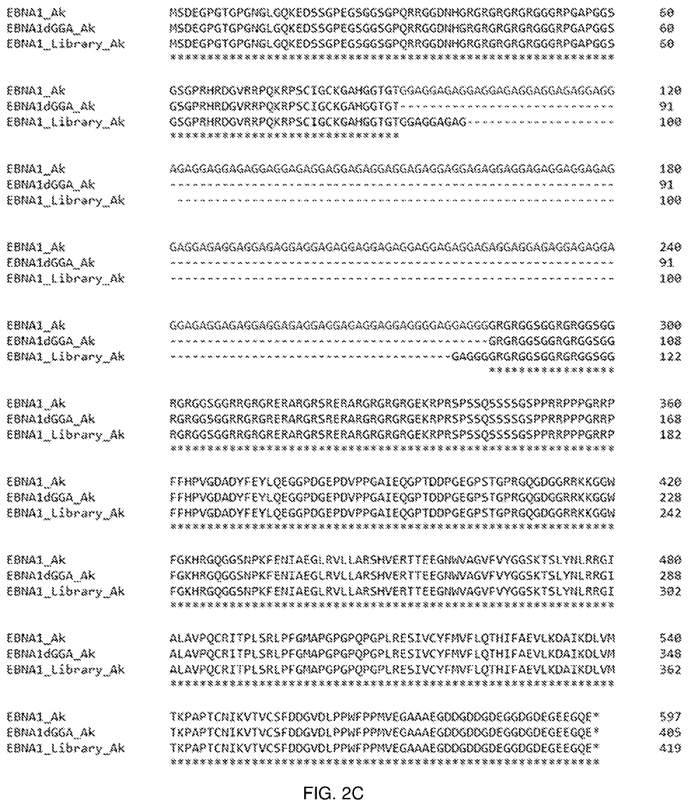

“dGGA-EBNA1” refers to an EBV EBNA1 protein in which a major portion of the Gly-Ala-rich region of the protein is deleted. As shown in FIGS. 1A and 1B, naturally-occurring EBNA1 protein comprises a region rich in, e.g. consisting solely of, the amino acids Gly and Ala extending from amino acid 90 to amino acid 327 in the context of FIG. 1A (EBNA1 from strain B95-8), or from amino acid 92 to amino acid 283 in EBNA1_Ak (FIG. 1B). In one example, the EBNA1 is that of the Akata strain, as depicted in FIG. 1B which provides a sequence of an Akata strain isolate EBNA1 protein. Useful dGGA-EBNA1 proteins (e.g., EBNA1dGGA_Ak and EBNA1_Library_Ak shown in FIG. 1B) for the detection methods and assays described herein have less than 50%, less than 40%, less than 30%, less than 25%, less than 20%, less than 15%, or less than 10% of the GA-rich region, that is, at least 50%, at least 60%, at least 70%, at least 75%, at least 80%, at least 85%, or at least 90%, including any increment therebetween, of the amino acids of the GA-rich region (e.g., amino acids from about amino acid 92 to about amino acid 283 in FIG. 1B), respectively, are deleted. As evident from the examples below, amino acids flanking the GA-rich region may also be deleted, so long as anti-EBNA1 IgA antibody can bind to the protein and the protein retains discriminatory ability with respect to NPC, as shown herein. One example of a useful modified EBNA1 is the EBNA1_Library_Ak identified in the examples below. That modified Akata EBNA1 protein comprises less than 10% of the GA-rich region, that is more than 90% of the GA-rich region is deleted (FIG. 1B). FIGS. 2A-2C provide alignments of the sequences of FIG. 1B, and a comparison of the respective sized of GA-rich regions FIG. 2B.

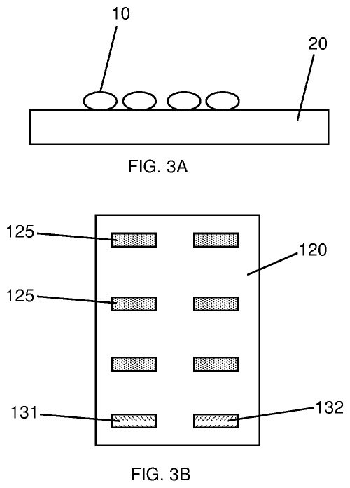

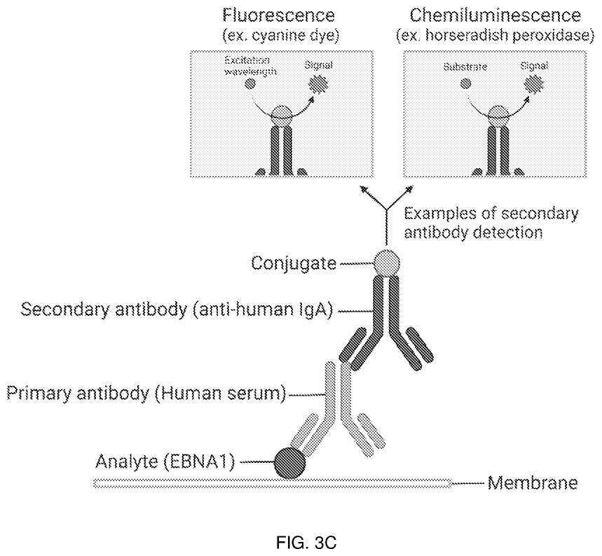

Provided herein is a test device and kit for use in identifying a patient's risk of developing NPG. Also provided is a method of treating a patient, comprising identifying the present of anti-EBNA1 IgA antibodies in a patient's blood, e.g., serum, and should anti-EBNA1 IgA antibodies be present in a patient's blood, monitoring the patient for development of NPC and treating the patient for NPC when present. The device comprises a dGGA-EBNA1 protein, e.g., a GAdel-Akata-EBNA1 protein, in which at least at least 50%, at least 60%, at least 70%, at least 75%, at least 80%, at least 85%, or at least 90% of the amino acids of the GA-rich region (e.g., amino acids from about amino acid 90 to about amino acid 328), are deleted. The dGGA-EBNA1 protein is attached to a protein-binding substrate (e.g., “substrate-bound”) at a discrete location on the substrate, with “protein-binding” and “substrate-bound” meaning a protein can be, or is, attached to, deposited on, or complexed, covalently, non-covalently, or otherwise affixed to the substrate at an identifiable location for purposes of the methods or assays described herein. The substrate can be a membrane, such as a nitrocellulose, nylon, or PVDF (polyvinylidene difluoride) membrane as is useful in Western, slot, and dot blotting protocols. The substrate can be any suitable substrate for immunogenic assays, such as a plastic or silicon surface, or a bead for use in flow cytometric assays, such as individually-addressable beads comprising individually-addressable fluorophore or quantum dot labels to which the target protein is bound. FIG. 3A depicts schematically the dGGA-EBNA1 protein 10 bound to the substrate 20. FIG. 3B depicts a membrane 120, an example of a substrate, having multiple discrete deposits of the dGGA-EBNA1 protein 125, and control deposits 131, 132, including purified human IgA as a positive control 131, and a negative control 132 with a bound protein different from the dGGA-EBNA1 protein to which anti-human IgA antibody cannot bind. FIG. 3C provides a schematic of an exemplary protein analyte immunodetection method. An analyte (EBNA1) is applied to a membrane. The membrane is incubated in a primary antibody (human serum) followed by a secondary antibody (anti-human IgA). The secondary antibody is conjugated to a detection molecule that can be visualized when excited at a specific wavelength (fluorescence) or by chemical reaction (chemiluminescence).

In one embodiment, a blot is used for detection of anti-EBV EBNA1 IgA antibodies in a patient's blood, such as in a serum or plasma sample. Blots, such as dot blots or slot blots, are simple Western-type blots, and their implementation is broadly described over the past decades, and involve detection of a protein analyte on a membrane with a primary antibody, followed by a secondary antibody which may be labeled with, for example and without limitation, a fluorescent moiety such as a cyanine dye, an enzyme for activating a colorimetric or chemiluminescent substrate such as horseradish peroxidase or alkaline phosphatase, or a radiolabel, such as ³⁵S. In the context of the present disclosure, seropositive anti-EBNA1 IgA serves as the primary antibody, with the secondary antibody being the anti-human IgA (See, e.g., FIG. 3C). Slot and dot bot techniques and implementation are broadly known for decades and need not be further described.

In the context of the present disclosure, blots may be produced by depositing lysate or purified protein solution onto as suitable membrane, such as a nitrocellulose membrane, a PVDF membrane, or any other suitable membrane to which a protein sample can be affixed and processed as described. Protein solutions can be applied directly in a small volume, or with a vacuum manifold to produce an orderly grid or array with separately addressable locations. Each dot or slot blot may contain known amounts of target protein or cell lysate, and an array of dots or slots may include positive and negative controls. Positive controls may include deposited human IgA_{H}, or the target protein to be detected by a positive sample with serum positive anti-EBNA1 IgA_{H} antibodies obtained either by synthetic generation of such antibodies, or from positive serum sample(s) obtained from a patient or multiple patients (pooled). Blots may be produced using commercially-available equipment, such as Bio-Dot® and Bio-Dot SF Microfiltration Apparatus (Bio-Rad Laboratories, Inc.) or PR 648 Slot Blot Manifold (Cytivia), among many others, production of suitable blots may be automated using fluidic and robotic devices. As above, blots, such as dot blots and slot blots may subjected to the same immunodetection steps used for Western blotting, e.g. blocking, antibody or patient test sample incubation, and target detection with substrate. For example, the EBNA1 proteins described herein may be deposited in one or more locations on a membrane. Once attached to the membrane, the membrane may be blocked according to standard Western blot practice to prevent non-specific binding to the membrane. The membrane may be washed between each detection step. A test sample may then be applied to a location of the deposited protein, such that any immunoglobulin may bind to the membrane-bound EBNA1 protein. One or more positive and/or negative control samples may be deposited to other spots or dots in the membrane (See, e.g., Example 3, below). The membrane may then be washed and then a solution comprising an anti-human IgA_{H} antibody may be contacted with one or more of the spots or dots. Again, positive and/or negative control samples may be used at this stage, too. The anti-human IgA_{H} antibody may be covalently (direct attachment) or non-covalently linked (e.g. using a suitable binding pair such as biotin/streptavidin binding) to a fluorophore, an enzyme, or a radiolabel, or other suitable tag useful in detection of any bound anti-human IgE antibody, as are broadly-known in the immunodetection arts. Likewise, the solution comprising an anti-human IgE linked to a tag may comprise a substrate for the tag, such as in the case of enzyme-labeled antibody detection, a colorimetric or chemiluminescent substrate for the enzyme, such as horseradish peroxidase (HRP) and one of its substrates, e.g. 3,3′-diaminobenzidine (DAB), 3,3′,5,5′-tetramethylbenzidine (TMB), and 2,2′-azino-di-[3-ethylbenzthiazoline-6-sulfonic acid] (ABTS) for colorimetric assays, or for chemiluminescence, H₂O₂ and luminol- and acridan-based reagents are chemiluminescent HRP substrates, as are broadly-known. HRP is a common enzyme used in such assays, but other enzymes, such as alkaline phosphatase, may be used similarly.

Blots may be housed in disposable housings that may be packaged as a detection device for single-use. The blot may be pre-blocked and dried, or may be provided “wet” within the package. The blot may be provided as a membrane with an indicia, bar code, QR code, or other markings to orient the blot and optionally upload data in a smartphone or scanner for detection using an app or cloud-based analysis and/or reporting computer-implemented processes. Reagents useful for detection of binding events may be packaged along with the detection device or blot, such as positive controls, optionally-labeled secondary anti-IgA antibodies, wash solutions, colorimetric or chemiluminescent substrates, dish(es) or tray(s) for use in processing the blot where a device is not used, and/or suitable liquid transfer devices, such as optionally disposable pipettes and syringes. Any reagent or the membrane may be provided in a lyophilized or dried state, which can be reconstituted prior to use. Sterile water may be provided in such a case. Alternatively, instead of a blot, the kit may comprise beads, optionally individually-addressable beads, for use in flow cytometry, and to which the described EBNA1 proteins are attached, along with, optionally, suitable control proteins or antibodies for positive and negative controls.

### EXAMPLES

The favorable prognosis of stage I and II nasopharyngeal carcinoma (NPC) has motivated a search for biomarkers for the early detection and risk assessment of Epstein-Barr virus (EBV)-associated NPC. Although EBV seropositivity is ubiquitous among adults, a spike in antibodies against select EBV proteins is a harbinger of NPC. A serological survey is provided that reveals which EBV antibodies could discriminate those at risk of developing NPC. Methods and data not shown or described herein are provided in priority application, U.S. Provisional Patent Application No. 63/336,590 filed Apr. 29, 2022, which is incorporated herein by reference in its entirety.

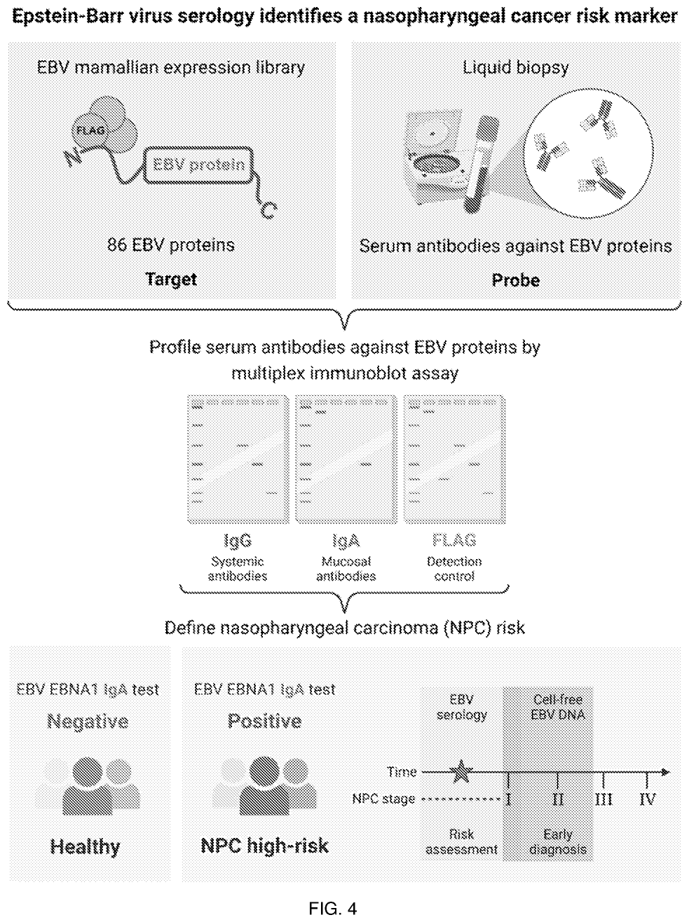

A comprehensive library of 86 EBV open-reading frames (ORFs) derived almost exclusively from the EBV Akata strain was developed to be expressed in mammalian cells. These mammalian lysates were used to conduct a serological survey for IgA, IgG, and IgM antibodies against EBV proteins, scored by a denaturing multiplex immunoblot, using serum from healthy individuals who were later diagnosed with NPC (incident cases) from a cohort of Singaporean Chinese, referred to as the Singapore Chinese Health Study (SCHS). These findings were validated in a similarly designed cohort study in Shanghai, China, named the Shanghai Cohort Study (SCS). Conventionally, an ELISA is the gold standard for measuring EBV serology, but ELISA is a low throughput assay that is not suitable for screening large numbers of EBV proteins. Because denaturing immunoblots using full-length EBV proteins are often used as a confirmatory assay, this labor-intensive but comprehensive multiplex approach provided a high degree of accuracy for screening a library of EBV proteins. Results showed a superior performance of serum IgA against EBNA1 (EBNA1 IgA) as the most informative biomarker for NPC risk assessment in case-control pairs of NPC pre-diagnostic sera. An overview of this study is provided in FIG. 4 and FIG. 1B provides exemplary sequences of polypeptides described in the Examples.

### Example 1

### Materials and Methods

Study Population: The SCHS is a residential cohort of 63,257 Chinese men and women from Singapore, aged 45-74 years at enrollment (1993-1998). Each subject was initially interviewed in person by trained personnel using a structured questionnaire including origin of province in China, dialect group (Cantonese and Hokkien), history of tobacco use, medical history, current diet, incense use at home, and for women, menstrual and reproductive history, and use of hormone replacement therapy. All blood components (plasma, serum, red blood cells, and white blood cells) were separated within 4-h post collection and stored at −80° C. Incident cancer cases among cohort participants were ascertained through record linkage with national databases of the Singapore National Cancer Registry and the Singapore National Birth and Death Registry, respectively. Since inception in 1993, <0.1% (56 subjects) were lost to follow-up by migration out of Singapore. As of Dec. 31, 2015, 50 participants who had no history of cancer were diagnosed with NPC, 42 of them with available baseline serum samples were included in this study. For each case, we randomly selected a control subject among all eligible participants who provided a baseline serum sample, were cancer-free and alive during the time from blood draw to cancer diagnosis of the index case and had no family history of NPC. The controls were followed for a median of 13 years (range 11.1-19.8), and none were diagnosed with NPC. The controls were individually matched to the index case by sex, dialect group (Hokkien, Cantonese), age at enrollment (±3 years), date of baseline interview (±2 years), and date of biospecimen collection (±6 months).

The SCS is a residential cohort of 18,244 men from Shanghai, China, aged 45-64 years at enrollment (1986-1989). Approximately 80% of eligible subjects agreed to participate in the study. Each subject was interviewed in person by trained personnel using a structured questionnaire including history of tobacco and alcohol use, current diet, and medical history. Blood samples were processed within 4-h after blood collection, and multiple aliquots of serum samples were stored at −70° C. or lower until analysis. Incident cases of cancer and death among the participants were identified via annual interviews and augmented by record linkage analysis with the datasets of the Shanghai Cancer Registry and the Shanghai Vital Statistics. The cohort follow-up is virtually complete; as of May 2019, 597 (3.3%) original participants refused to continue, and 754 (4.1%) were lost to annual follow-up due to change of residence or migration out of Shanghai. As of 2019, 37 participants who were cancer-free at baseline were diagnosed with NPC and included in this study. Similarly, one control subject was randomly chosen for each case. The control was matched to the index case by age (±2 years), date of blood draw (±1 month), and the same neighborhood of residence at study enrollment. The controls were followed for a median of 28.6 years (range 7.6-31.6), and none were diagnosed with NPC.

EBV Expression Library: Out of 87 possible EBV ORFs, 86 were cloned to generate a new EBV ORF mammalian expression library. This library was based primarily on the EBV Akata genome (83/86 ORFs) (Lin Z, et al. Whole-Genome Sequencing of the Akata and Mutu Epstein-Barr Virus Strains. Journal of Virology 2013; 87(2):1172-82). Eighty-five ORFs were cloned, and one ORF (BPLF1 derived from EBV B95-8) was a gift. LF3 was the only ORF that could not be cloned or synthesized due to repetitive sequences. Seventy-four ORFs were PCR-amplified with Phusion High-Fidelity DNA Polymerase (Thermo Scientific) using primers based on the EBV Akata strain (GenBank KC207813.1), and one ORF (LMP2A) spanning the terminal repeats was based on the EBV B95-8 strain due to available cDNA (GenBank V01555.2). Ten ORFs were codon optimized and synthesized (Genewiz, Azenta), nine of which were based on EBV Akata, and one ORF (BHLF1) was based on EBV B95-8 because it is truncated in the Akata genome. Genomic DNA extracted from Akata B-cells with the GENEJET Genomic DNA Extraction Kits (Thermo Scientific) served as template for amplification. For the large ORF LMP2A, RNA was extracted with the GENEJET RNA kit and cDNA was synthesized using Revert Aid First Strand cDNA Synthesis Kit (Thermo Scientific). The PCR products were cloned in-frame with the 3X FLAG-tag of a modified mammalian expression vector. The modified vector was generated by inserting a linker sequence (5′-AAT TCG CGA TCG CTT AAT TAA ACC ATG GAC TAC AAA GAC CAT GAC GGT GAT TAT AAA GAT CAT GAC ATC GAT TAC AAG GAT GAC GAT GAC AAG CTT GCG GCC GCG GTA CCG-3′ (SEQ ID NO: 5)) into EcoRI and Sall sites of the p3XFLAG-CMV-7 mammalian expression vector (Sigma-Aldrich). EBV ORF BPLF1 was cloned into pFLAG-CMV-2 (Sigma-Aldrich). All cloned EBV ORFs were sequenced-verified.

Two ORFs (BKRF1 (EBNA1), BYRF1 (EBNA2)) showed some size variation relative to the Akata sequence. PCR-amplified EBNA1 was shorter than expected but maintained an intact C-terminus including amino acids 408-498 which is reported to be immunodominant. The size difference resulted from a 534-bp (amino acids 98-275) deletion of the Gly/Ala-repeat variable region. EBNA2 was shorter by 111-bps within the 120-bp N-terminal polyproline repeat. All cloned EBV ORFs were fused in-frame to encode a FLAG-tag at the N-terminus for normalization by FLAG expression. Immunoblot detection values above 293 cell background levels were observed for all transfected EBV proteins.

Cell Lines and Preparation of Lysates: The EBV-positive B-cell line, Akata clone 21, was maintained in RPMI-1640 medium supplemented with 10% fetal bovine serum (FBS). HEK293 cells (CRL-1573, ATCC) were grown in DMEM supplemented with 10% FBS. Cell lines are routinely screened (at least once every 2 months) for mycoplasma contamination by PCR and validated by DNA fingerprint using Short Tandem Repeat profiling. Thawed cells were maintained for a maximum of 15-20 passages. Transfections of the 293 cells were performed using X-tremeGENE 9 DNA Transfection Reagent (Roche). Whole cell lysates were prepared in radioimmunoprecipitation assay (RIPA) lysis buffer supplemented with 1 mM phenylmethylsulfonyl, 2 mM activated sodium orthovanadate, a 1:100 dilution of protease inhibitor and phosphatase inhibitor cocktails (Sigma-Aldrich), clarified by centrifugation, aliquoted, and stored at −80° C.

Multiplex Immunoblot Assay: Whole-cell lysates were prepared by boiling or incubation at 70° C. in 1× protein sample loading buffer (LICOR) supplemented with 2.5% β-mercaptoethanol. Purified human whole IgG, IgA, IgM (ChromPure, Jackson IR), and FLAG-BAP recombinant protein (Sigma-Aldrich) were used as positive loading controls and for normalization of detected signals in each gel. Lysates were separated by SDS-PAGE in 4-20% acrylamide gradient gels (Bio-Rad Criterion TGXTM) and transferred to 0.2 μm nitrocellulose membrane (Bio-Rad Trans Blot Turbo). Membranes were blocked in 5% nonfat dry milk in Tris-buffered saline (TBS) and incubated overnight at 4° C. with a 1:500 dilution of pre-adsorbed human sera and 1 μg/ml anti-FLAG M2 clone primary antibody (AB_259529, Sigma-Aldrich). Serum pre-absorption was carried out prior to primary incubation for 24-h at 4° C. in 293 cells that were previously fixed in 4% paraformaldehyde and permeabilized in 0.1% Triton X-100. After primary incubation, membranes were washed in TBS (3×5-min) followed by 1-hour incubation with fluorescently tagged secondary antibodies at room temperature in the dark, diluted in blocking buffer (3.75 μg/ml Cy3-AffiniPure goat anti-hIgAH (AB_2337721); 0.03 μg/ml AlexaFluor 680-AffiniPure goat anti-hIgGH (AB_2889013); 3.75 μg/ml Cy3-AffiniPure goat anti-hIgMH (AB_2337729, Jackson IR); 0.1 μg/ml IRDye680LT goat anti-hIgGH+L (AB_10795013); 0.1 μg/ml IR800CW goat anti-mIgGH+L (AB_621842, LICOR). Each membrane was incubated with an optimized dilution of a mixture of anti-mIgGH+L, anti-hIgAH and anti-hIgGH; or anti-mIgGH+L, anti-hIgGH+L and anti-hIgMH antibodies which were validated in house to ensure no cross reactivity between secondary antibody binding or channel fluorescence. The subscript H or H+L indicate the target “heavy” or “heavy and light” chain, respectively. Targeting the heavy chain is antibody specific because each class of antibody contains a unique heavy chain, whereas targeting the light chain that is common to all antibodies provides a non-specific antibody marker and indicates the total antibody immune response.

The membranes were scanned on a Bio-Rad ChemiDoc MP scanner, and the levels of detection in three channels were quantified using ImageLab 6 software (Bio-Rad). The serum of EBV seropositive and seronegative healthy individuals with known serologic spectra were used to initially validate the multiplex assay before quantifying SCHS and SCS samples. A result was determined to be positive when a band (1) appeared at the expected size in the IgG, IgA, or IgM channel, (2) matched the protein band found in the FLAG channel, (3) was absent in the untransfected HEK293 cell lysate lane, and (4) was above the background level of the HEK293 cell lysate lane. The “normalized ratio” of values detected in the 700 nm, 500 nm channels (representing protein detection levels by human sera) divided by values in the 800 nm channel (representing EBV ORF protein expression levels by FLAG), were calculated, and arranged in a heatmap using Prism 9 software (GraphPad, La Jolla, CA). Personnel running and scoring the immunoblots were blinded to the identity and case/control status of sera. EBV ORFs listed as putative were determined by previous studies and cross referenced with reviewed UniProt submissions to determine proteins without evidence at the protein level. According to these criteria, the putative ORFs were RPMS1, A73, BARF0, and BWRF1, and our results detected antibodies against A73 (IgGH+L), BARF0 (IgGH, IgGH+L), and BWRF1 (IgGH) of which at least one of the detected bands was the predicted molecular weight or larger.

HH514-16 cells (Rabson M, et al. Identification of a rare Epstein-Barr virus variant that enhances early antigen expression in Raji cells. Proc Natl Acad Sci USA 1983; 80(9):2762-6) were grown in 10% FBS-supplemented RMPI-1640 media to 1×10⁶ cells/ml and induced by 3 mM sodium butyrate (NaB) for 48 h. Lysates were harvested, resolved by SDS-PAGE, and transferred to nitrocellulose membranes by the same protocol described for the EBV ORF expression library. Purified whole IgA was run as a positive loading control and lysate from uninfected Akata B-cells served as a negative control. Primary and secondary antibody incubations were identical to the methods described for the EBV ORF expression library, without M2 primary antibody. The number of bands detected (band count) were scored by an independent evaluator blinded to the sample identity.

Conservation Plots: Publicly available whole genome assemblies of EBV (n=127) were extracted from NCBI in FASTA format and categorized based on; tissue origin, endemicity, and geographical origin. The EBNA1 gene region was extracted, and the coding region translated using an in-house Python script until an in-frame stop codon was detected. The amino acid sequences were aligned using the Clustal Omega multiple sequence alignment tool. Each of the multiple sequence alignments were manually curated to stretch a length of the reference 642 amino acids. Uncertain amino acids (designated “X”) were removed within the Glycine-Alanine repeat region. Conservation was calculated based on EBV Akata or B95-8 reference sequences and visualized using R.

Statistical Analysis: x² (for categorical variables) or t-test (for continuous variables) statistics were used to examine the difference in the distributions of selected baseline demographic and lifestyle variables between NPC cases and controls. Conditional logistic regression method was used to estimate odds ratio and 95% confidence intervals of NPC development for the EBNA1 IgA detection. Fisher's exact test was used to examine the difference in proportion of serum samples detecting each EBV protein between cases and controls. A Mann-Whitney test was used to compare the numbers of EBV proteins (nEBV) detected in NPC cases to those in controls. Sparse logistic regression analysis was used to compare the EBV antibody response rates 259 in NPC cases and controls by fitting a logistic regression with the L1 regularization (25). The L1 regularization forces the model weights of unimportant immunoreactivity values to be zero, resulting in a sparse logistic model. The scalar multiplier for the L1 penalty term was set to 0.5. A log-likelihood ratio test of the fitting result was performed to determine the predictive significance of EBV protein classes between NPC cases and controls. Statistical analyses were carried out using SAS software version 9.4 (SAS Institute, Cary, NC), the statsmodels package in Python, and Prism 9. The p values of less than 0.05 were statistically significant.

### Results

Out of 27 pre-diagnostic serum samples collected within 8 years to diagnosis from the endemic SCHS cohort, 20 case-control pairs were prioritized based on sufficiently available sample to screen the entire EBV ORF library (discovery group). Among the 86 EBV proteins tested for IgA_{H} (heavy chain-specific) in these sera, EBNA1 IgA was the most informative biomarker such that 19 of the 20 NPC cases, and none of the 20 matched controls tested positive (p<0.0001). All values were determined by immunoblot densitometric analysis and reported as a ratio of the antibody isotype-specific detection value divided by the FLAG-tagged EBV protein detection value as defined in the methods.

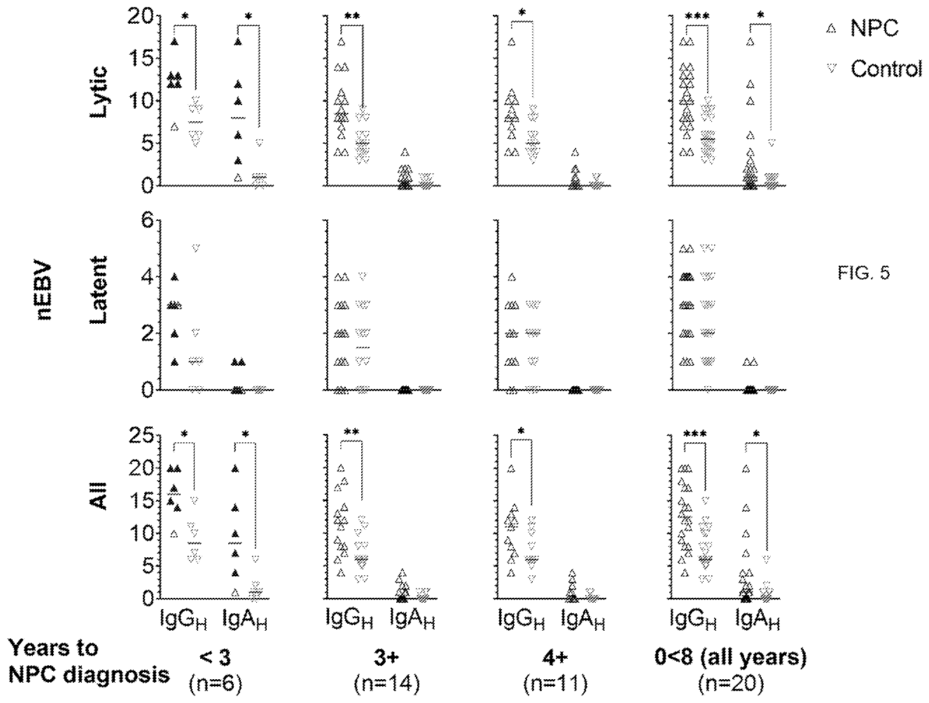

EBNA1 is classified as a latent protein but is an essential protein that is produced in all types of EBV infection. The single incident NPC (case ID: NPC12) negative for EBNA1 IgA showed no IgA reactivity to any EBV protein. This patient was a heavy cigarette smoker (23 pack-years) and the heaviest drinker (6 drinks/day) among the 20 pairs tested. It is unclear if this NPC case was EBV-associated, and we do not have access to tumor tissue for confirming EBV status by EBV-encoded small RNA (EBER) staining. The IgG response indicates that the patient was EBV seropositive. Excluding EBNA1 IgA, 18 of the 26 EBV proteins detected by IgA were unique to incident NPC cases which were named “NPC differential IgA panel” (FIG. 3), and 14 of these appeared only in NPC cases within 4 years to diagnosis (case IDs: NPC1-NPC9) (FIG. 5). For three EBV proteins, IgA detection was enriched in the 20 cases compared to controls including, BFRF3 (12 cases vs 4 controls, p=0.0098), BKRF4 (4 cases vs 0 controls, p=0.0350), and BALF2 (4 cases vs 0 controls, p=0.0350), but combinations of these or other markers did not improve upon the superior EBNA1 IgA test score.

Other immunoglobulins (IgG_{H}, IgM_{H} heavy chain-specific) were evaluated for NPC predictive biomarkers. A third parameter, IgG heavy and light chain (IgG_{H+L}), was scored as total antibody response due to the shared light chains between antibody isotypes. There were notable high ranking IgG targets such as BNLF2b IgG that discriminated NPC cases from controls (12 cases vs 1 control, p=0.0002), which is consistent with results for BNLF2b IgG_{H+L} (15 cases vs 1 control, p<0.0001), but none of these outperformed EBNA1 IgA. IgM, the antibody isotype produced in abundance during primary exposure, showed no value for risk assessment because it was detected in only one case (case ID: NPC1). A sparse logistic regression affirmed that EBNA1 IgA and BNLF2b IgG were the top-ranking discriminators of incident NPC by the greatest assigned positive coefficients (weight: EBNA1 IgA 5.4198, BNLF2b IgG 3.9207). In contrast, a negative weight would indicate discriminatory antibodies in the control group, which could reveal protective antibodies against NPC, but none were comparable in magnitude. We also evaluated EBV proteins detected by IgA or IgG for discriminating NPC cases closer to diagnosis (within 4 years) from those collected at longer time intervals to diagnosis but did not identify a candidate (all p>0.05). When data were analyzed for the aggregated numbers of EBV proteins detected by IgA or IgG other than EBNA1, it was found that IgG detected a greater number of lytic proteins in cases than controls regardless of time interval from serum collection to NPC diagnosis (FIG. 5). Additionally, IgA detected a greater number of lytic proteins in cases than controls but only in sera that was collected within 3 years to NPC diagnosis (FIG. 5). While this did not identify a specific biomarker that could discriminate cases closer to diagnosis, the data revealed an important characteristic of EBV pathogenesis. The expanded EBV IgA repertoire is consistent with the hypothesis that the spread of EBV-infected cells (epithelial or B-cells) at mucosal sites is a distinguishing feature of those at imminent risk of developing NPC.

Based on these findings, EBNA1 IgA was focused on as the leading NPC predictive biomarker. The test for EBNA1 IgA was expanded to 22 additional incident NPC cases from the SCHS (median 8.84, range 0.43-14.3 years to diagnosis) (FIG. 1). Overall, EBNA1 IgA sensitivity was 85.7% (36/42 cases) and specificity was 92.9% (3/42 false-positives) (Table 1). When the data was stratified by years to NPC diagnosis, all 13 sera collected within 4 years to NPC diagnosis were positive for EBNA1 IgA whereas the matched control sera were negative (Table 1). We conclude that EBNA1 IgA has a sensitivity of 100% within 4 years to diagnosis, and an overall specificity of 92.9% for all controls tested in the SCHS cohort. Table 1 provides data re detection of EBNA1 IgA in pre-diagnostic sera of study participants who developed NPC and those who remained cancer free (controls), The Singapore Chinese Health Study (SCHS).

TABLE 1

| EBNA1 status by | NPC cases | Matched controls | Fisher exact test |
|---|---|---|---|
| time to diagnosis | (%) | (%) | 2-sided P |
| All | | | |
| Positive | 36(85.7) | 3 (7.1) | 8.98 × 10⁻¹⁴ |
| Negative | 6(14.3) | 39 (92.9) | |
| 0 to 3.99 years | | | |
| EBNA1 pos | 13 (100.0) | 0 (0.0) | 1.92 × 10⁻⁹ |
| EBNA1 neg | 0 (0.0) | 13 (100.0) | |
| 4 or more years | | | |
| EBNA1 pos | 23 (79.3) | 3 (10.3) | 1.61 × 10⁻⁹ |
| EBNA1 neg | 6 (20.7) | 26 (89.7) | |

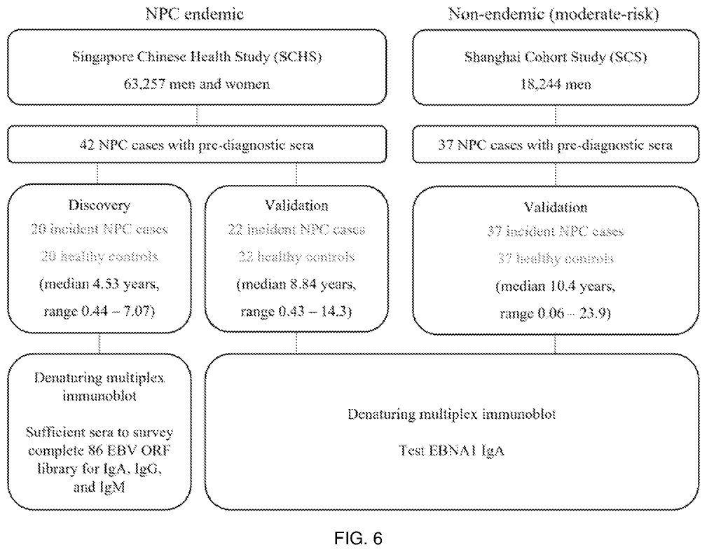

EBNA1 IgA was then tested in sera from participants of the SCS, an independent prospective cohort study in Shanghai, China, where NPC incidence rates were lower than in Singapore. We identified 37 incident NPC cases with pre-diagnostic sera (median 10.4 years, range 0.1-23.9) (FIG. 6). EBNA1 IgA sensitivity was 59.5% (22/37 cases) and specificity was 67.6% (12/37 false-positives) (Table 2). Because the SCS focused on cancer diagnosis and risk factors, information on other EBV-related co-morbidities (such as chronic herpesvirus reactivation, immune suppression, autoimmunity, or acute EBV infection) that could have explained the EBNA1 IgA false positive results, were not collected. Interestingly, all 7 sera from participants diagnosed with NPC within 4 years were positive for EBNA1 IgA and all 7 matched controls were negative (Table 2). This confirmed that EBNA1 IgA has a sensitivity of 100% within 4 years to diagnosis in both the SCHS and SCS case-control cohorts. Table 2 provides data showing detection of EBNA1 IgA in pre-diagnostic sera of study participants who developed NPC and those who remained cancer free (controls), The Shanghai Cohort Study (SCS).

TABLE 2

| EBNA1 status by | NPC cases | Matched controls | Fisher exact test |
|---|---|---|---|
| time to diagnosis | (%) | (%) | 2-sided P |
| All | | | |
| Positive | 22 (59.5) | 12 (32.4) | 3.5 × 10⁻² |
| Negative | 15 (40.5) | 25 (67.6) | |
| 0 to 3.99 years | | | |
| EBNA1 pos | 7 (100.0) | 0 (0.0) | 5.8 × 10⁻⁴ |
| EBNA1 neg | 0 (0.0) | 7 (100.0) | |
| 4 or more years | | | |
| EBNA1 pos | 15 (50.0) | 12 (40.0) | 6.0 × 10⁻¹ |
| EBNA1 neg | 15 (50.0) | 18 (60.0) | |

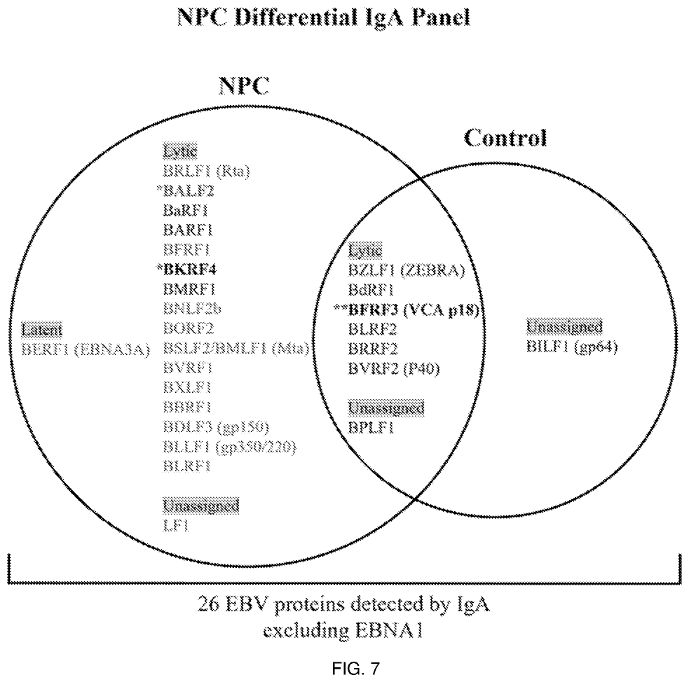

Whether a combination of EBV-specific IgA could reduce the false positive rate of the EBNA1 IgA test was examined. EBV IgA proteins that differentiated NPC cases (FIG. 7) that were detected more than once within 4 years to diagnosis were focused on. From the initial EBV library screen performed on the 20 case-control pairs from the SCHS discovery group, analysis was expanded on the remaining 31 pairs from both SCHS and SCS whose sera were collected within 10 years to diagnosis. However, these additional eight EBV proteins (EBNA3A, EA p138, BFRF1, BNLF2b, BORF2, SM, TK, BLRF1) that best discriminated incident cases from controls did not increase the specificity (false positive rate) of EBNA1 IgA alone, nor did they discriminate cases by time (<4 years vs 4+ years). Due to the limited serum samples available, we were not able to test the whole EBV library panel against these additional samples, but were able to extend the IgA analysis against lytic proteins with the additional specimens using an alternative method. Immunoblots performed on EBV-reactivated cell lysates from HH514-16 cells is an established method for scoring IgA and IgG molecular diversity against EBV lytic proteins. It was found that IgA detection of lytic proteins could indeed distinguish NPC cases by time (p<0.05, <3 years vs. 3+ years to diagnosis), with no difference in the controls. This confirms that IgA diversity is increased in NPC cases closer to diagnosis and this feature might be exploited in the future to stratify EBNA1 IgA test-positive cases by time.

The use of EBNA1 IgA for NPC early detection and risk assessment has been evaluated by numerous studies. Strikingly, incident NPC cases are identified herein with 100% sensitivity and 100% specificity 4 years before clinical manifestation when control subjects were matched according to pre-defined criteria. When extended to all control subjects, the specificity was 92.9% in the endemic cohort (SCHS) and 67.6% in a lower risk cohort (SCS).

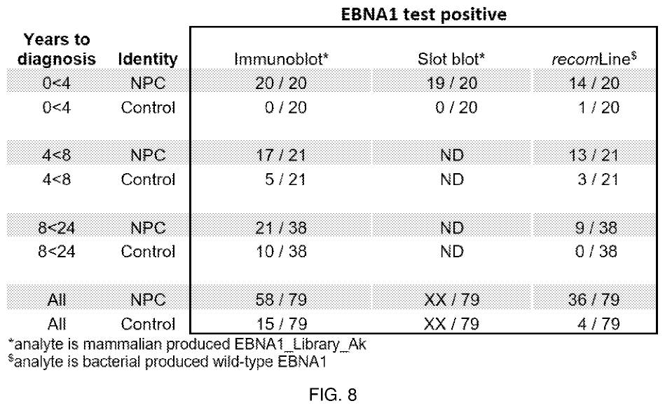

The superior performance of EBNA1 IgA herein may be attributed to the choice in methodology and the EBV strain. Immunoblotting is typically used as a confirmatory assay for ELISA-based tests for early diagnosis of NPC. Immunoblots can deliver a high degree of sensitivity and specificity, as demonstrated by the original human immunodeficiency virus (HIV) molecular diagnostic test used in clinical pathology. The use of multiplex immunoblots enables the denaturation and size resolution of EBV proteins which greatly reduces the false-positives and false-negatives that are commonly found in semi-denaturing methods (such as protein array and ELISA-based assays). In terms of EBV strain, the prototypic B95-8 strain is often used in serology assays. Both Akata and B95-8 strains are classified as Type I EBV, but the Akata strain shares greater sequence similarity and identity with EBV strains found in NPC tumors. The Akata EBNA1 amino acid sequence is almost identical to EBNA1 from NPC tumors whereas the B95-8 EBNA1 amino acid sequence is dissimilar from NPC tumors in at least 3 residues in the immunodominant epitopes (IDE, amino acids 382-410, 413-452). The expression system also was considered, as prior studies have used bacterial expression systems, peptide microarrays, or in-vitro translated cell-free expression systems. Compared to these alternative methods, the EBV mammalian expression library described herein produces FLAG-tagged full-length EBV proteins in human cells which best mirror the post-translational modifications of EBV proteins in vivo. Upon refinement of test parameters, it can be possible to scale this assay to a clinical setting in the format of a rapid test (see, e.g., Paramita DK, et al. Evaluation of commercial EBV RecombLine assay for diagnosis of nasopharyngeal carcinoma. Journal of Clinical Virology 2008; 42(4):343-52). Comparison with the commercial assay is shown in FIG. 8.

Increased EBV lytic gene expression and expansion of the IgA repertoire against EBV are thought to immediately proceed NPC development. This is thought to originate from circulating EBV-reactivated B-cells that infiltrate nasopharyngeal epithelia and/or EBV reactivation in infected epithelial cells in response to cellular differentiation in the stratified epithelium. These reactivated cells can be abortive and therefore the diversity of induced EBV proteins can vary widely from cell to cell as evidenced by single cell RNA-sequencing. Thus, it is not clear if a specific assortment of lytic EBV proteins can discriminate incident NPC cases from healthy individuals. However, EBNA1, which is expressed in all types of EBV infection, was consistently detected by IgA within 4 years in both cohorts (100% sensitivity), and only showed a precipitous drop in detection in samples greater than 10 years to diagnosis. Although EBNA1 IgA is present in all seropositive individuals, we postulate that the assay described herein detects elevated EBNA1 IgA, most likely reflecting heightened EBV immunity at the nasopharyngeal mucosa before the onset of NPC.

In the later time frame (>4 years to diagnosis), the SCS cohort appears to have reduced discrimination by EBNA1 IgA, but this could be explained because most samples were collected beyond 10 years to diagnosis. At present, it is not clear why more SCS control samples tested positive for EBNA1 IgA in this latter time frame than in the endemic SCHS cohort. These individuals may have an EBV-related comorbidity with uncontrolled EBV levels and immunity. Furthermore, identifying an additional parameter that stratifies EBNA1 IgA test-positives into imminent cases would be important. While an increased IgG repertoire was detected some years before diagnosis (4+ years), the increased IgA repertoire was limited to 2 years before diagnosis. This finding adds to our knowledge of EBV immune surveillance in the pathology of NPC. One scenario consistent with these observations would be that persons at risk of developing NPC are distinguished by chronically high levels of EBV lytic infection in their blood some years before NPC presentation, which would traffic to mucosal sites to result in high levels of lytic infection at the nasopharyngeal mucosa a few years before NPC presentation.

Tackling the issue of cancer prevention requires a holistic approach. Towards this goal, EBV serology may be combined with EBV DNA analysis to discriminate EBNA1 IgA test-positives already at early stage NPC. The short half-life of plasma cell-free EBV DNA tracks remarkably well with radiotherapy and the response of the NPC tumor. Because the source of the cell-free EBV DNA is likely tumor-derived, its potential as a risk assessment marker may be limited although this remains to be thoroughly examined. Upon implementation of a population screen, a clear clinical follow-up by MRI or endoscopy would have to be defined for test-positives. Given that HIV-positive patients treated with ganciclovir reduced the risk of Kaposi's sarcoma by 93%, it may be possible that antiviral therapy, e.g., acyclovir, valaciclovir, and/or tenofovir, may be used prophylactically. An EBNA1 inhibitor effective at restricting NPC tumor cell growth in a xenograft model is in clinical trials. At present an EBNA1 IgA risk assessment test would provide individuals identified as high-risk an opportunity for regular follow-up by clinical examination and endoscopy, though effective prophylaxis would be warranted.

### Example 2

### Materials and Methods

### Study Population

Singapore Chinese Health Study (SCHS): The SCHS is a residential cohort of 63,257 Chinese men and women from Singapore, aged 45-74 years at enrollment (1993-1998). Each subject was initially interviewed in person by trained personnel using a structured questionnaire including origin of province in China, dialect group (Cantonese and Hokkien), history of tobacco use, medical history, current diet, incense use at home, and for women, menstrual and reproductive history, and use of hormone replacement therapy. All blood components (plasma, serum, red blood cells, and white blood cells) were separated within 4-h post collection and stored at −80° C. Incident cancer cases among cohort participants were ascertained through record linkage with national databases of the Singapore National Cancer Registry and the Singapore National Birth and Death Registry, respectively.

Shanghai Cohort Study (SCS): The SCS is a residential cohort of 18,244 men from Shanghai, China, aged 45-64 years at enrollment (1986-1989). Approximately 80% of eligible subjects agreed to participate in the study. Each subject was interviewed in person by trained personnel using a structured questionnaire including history of tobacco and alcohol use, current diet, and medical history. Blood samples were processed within 4-h after blood collection, and multiple aliquots of serum samples were stored at −70° C. or lower until analysis. Incident cases of cancer and death among the participants were identified via annual interviews and augmented by record linkage analysis with the datasets of the Shanghai Cancer Registry and the Shanghai Vital Statistics.

TABLE 3
Baseline demographic and lifestyle characteristics of study participants
who developed NPC (cases) and those who remained cancer free (controls)

| | SCHS | | | SCS | | |
|---|---|---|---|---|---|---|
| Characteristic | Case | Control | p^{a} | Case | Control | p^{a} |
| Subjects | 42 | 42 | | 37 | 37 | |
| Age (year), mean ± SD | 59.0 ± 6.1 | 58.9 ± 6.1 | 0.957 | 53.9 ± 5.3 | 54.0 ± 5.1 | 0.937 |
| Body mass index, kg/m² | 23.1 ± 3.0 | 23.7 ± 3.2 | 0.380 | 22.2 ± 3.5 | 21.9 ± 2.7 | 0.676 |

Highest level of education, %

| | | | | | | |
|---|---|---|---|---|---|---|
| No formal education | 7.1 | 2.4 | | 8.1 | 2.7 | |
| Primary school | 47.6 | 40.5 | | 16.2 | 21.6 | |
| Secondary school or above | 45.3 | 57.1 | 0.402 | 75.7 | 75.7 | 0.641 |

Cigarette smoking, %

| | | | | | | |
|---|---|---|---|---|---|---|
| Never smokers | 47.6 | 57.1 | | 43.2 | 45.9 | |
| Former smokers | 16.8 | 23.8 | | 0 | 5.4 | |
| Current smokers | 35.6 | 19.1 | 0.221 | 56.8 | 48.7 | 0.480 |

Alcohol drinking, %

| | | | | | | |
|---|---|---|---|---|---|---|
| Non-drinkers | 69.0 | 64.3 | | 73.0 | 54.1 | |
| Moderate drinkers | 26.2 | 35.7 | | 18.9 | 29.7 | |
| Heavy drinkers^{b} | 4.8 | 0.0 | 0.261 | 8.1 | 16.2 | 0.293 |

^{a}2-sided p values were based on t test for continuous variables or chi-square test for categorical variables;
^{b}Heavy drinking was defined as >3 drinks/day for men and >2 drinks/day for women, and lower levels as moderate drinking.

### Immunoblot Assay by Slot Blot

Immunoblot: Bio-Dot SF Microfiltration Apparatus (Bio Rad 1706542); Nitrocellulose membrane (Bio Rad 1620112); Slot blot filter paper (Bio Rad 1620161).
Control proteins for loading input and antibody detection: Amino terminal FLAG-BAP fusion protein (Sigma P7582-100UG); ChromPure IgA whole molecule (Fisher NC0004397); ChromPure IgG whole molecule (Fisher NC9701046)
Antibodies: Primary—Monoclonal mouse anti-FLAG clone M2 (Sigma F1804); Secondary—Affinipure Goat Anti-Human Serum IgA 2.0 mg (Fisher NC1618588) *detects human IgA; Secondary—AffiniPure Alexa 680 Goat Anti-Human IgG 0.5 mg (Fisher NC1969399) *detects human IgG; Secondary—Li-Cor IRDye 800CW Goat Anti-Mouse IgG(H+L) 0.5 mg (Fisher NC9401841) *detects FLAG M2
Biospecimens (pre-diagnostic NPC serum): See “Study Population” published materials and methods.

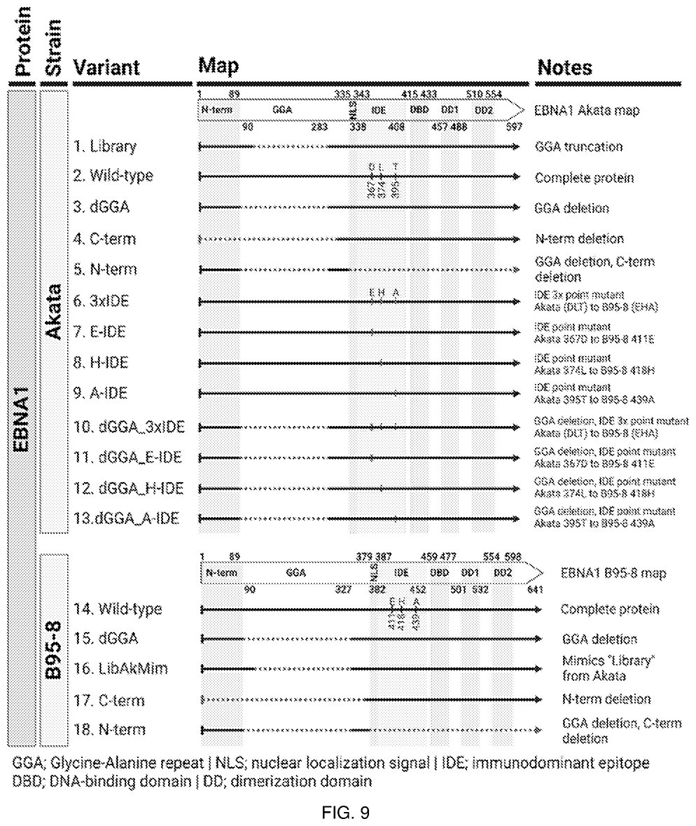

EBNA1 constructs: Table 4 lists EBNA1 constructs that have been created. These constructs and derivatives of EBNA1_dGGA_Ak and EBNA1_Library_Ak, will be used to interrogate NPC risk. In Table 4, EBNA1 constructs from EBV strain Akata were derived from the genomic consensus sequence for the EBV genome Genbank no. KC207813.1 (AFY97842.1 for EBNA1 amino acid sequence). EBNA1 constructs from strain B95-8 were derived from the consensus sequence for the EBV genome Genbank no. V01555.2 (CAA24816.1 for EBNA1 amino acid sequence). Constructs expressing naturally occurring (wild-type) variants of EBNA1 were cloned by PCR from EBV genomic DNA or by gene synthesis. Derivative constructs that are not derived from naturally occurring sequences were generated by gene synthesis. Deletion and point mutants were engineered by PCR cloning or by In-Fusion cloning (Takara Bio). All EBNA1 constructs were cloned in-frame with an N-terminal 3xFLAG-tag. FIG. 9 provides a graphical depiction of EBNA1 constructs from Table 4.

TABLE 4

| | | | Truncation, | |
|---|---|---|---|---|
| | | | Deletion or | |
| | EBNA1 Construct | | Point | Specific |
| # | Name | Strain | Mutation | Modification(s) |
| 1 | EBNA1_Library_Ak | Akata | Gly/Ala | aa 101-277 deleted |
| | (SEQ ID NO: 4) | | truncation | |
| 2 | EBNA1_Ak | Akata | — | Same as consensus |
| | (SEQ ID NO: 2) | | | sequence |
| | | | | (AFY97842.1) |
| 3 | EBNA1dGGA_Ak | Akata | Gly/Ala | aa 92-283 deleted |
| | (SEQ ID NO: 3) | | deletion | |
| 4 | EBNA1_C-term_Ak | Akata | N-term + | aa 1-345 deleted |
| | | | Gly/Ala | |
| | | | deletion | |
| 5 | EBNA1_N-term_Ak | Akata | C-term + | aa 92-283 deleted |
| | | | Gly/Ala | and aa 346-597 |
| | | | deletion | deleted |
| 6 | EBNA1_3xIDE_Ak | Akata | IDE point | IDE residues |
| | | | mutations | changed from Akata |
| | | | | to B95-8 sequence |
| | | | | at aa 367 (D to E), |
| | | | | 374 (L to H), 395 |
| | | | | (T to A) |
| 7 | EBNA1_E-IDE_Ak | Akata | IDE point | aa 367 D (Akata) to |
| | | | mutant | E (B95-8) |
| 8 | EBNA1_H-IDE_Ak | Akata | | aa 374 L (Akata) to |
| | | | | H (B95-8) |
| 9 | EBNA1_A-IDE_Ak | Akata | | aa 395 T (Akata) to |
| | | | | A (B95-8) |
| 10 | EBNA1dGGA_{—} | Akata | Gly/Ala | aa 92-283 deleted, |
| | 3xIDE_Ak | | deletion + | IDE residues |
| | | | IDE point | changed from Akata |
| | | | mutations | to B95-8 sequence |
| | | | | at aa 367 (D to E), |
| | | | | 374 (L to H), 395 |
| | | | | (T to A) |
| 11 | EBNA1dGGA_E- | Akata | Gly/Ala | aa 92-283 deleted, |
| | IDE_Ak | | deletion + | aa 367 D (Akata) to |
| | | | IDE point | E (B95-8) |
| 12 | EBNA1dGGA_H- | Akata | mutant | aa 92-283 deleted, |
| | IDE_Ak | | | aa 374 L (Akata) to |
| | | | | H (B95-8) |
| 13 | EBNA1dGGA_A- | Akata | | aa 92-283 deleted, |
| | IDE_Ak | | | aa 395 T (Akata) to |
| | | | | A (B95-8) |
| 14 | EBNA1_B9 | B95-8 | — | Same as consensus |
| | (SEQ ID NO: 1) | | | sequence |
| | | | | (CAA24816.1) |
| 15 | EBNA1dGGA_B9 | B95-8 | Gly/Ala | aa 90-327 deleted |
| | | | deletion | |
| 16 | EBNA1_LibAkMim_{—} | B95-8 | Gly/Ala | Mimics |
| | B9 | | truncation | EBNA1_Library_{—} |
| | | | | Ak (#1), aa |
| | | | | 101-321 deleted |
| 17 | EBNA1_C-term_B9 | B95-8 | N-term + | aa 1-389 deleted |
| | | | Gly/Ala | |
| | | | deletion | |
| 18 | EBNA1_N-term_B9 | B95-8 | C-term + | aa 90-327 deleted, |
| | | | Gly/Ala | aa 390-641 deleted |
| | | | deletion | |

aa; amino acid, dGGA; Gly/Ala deletion, C-term; carboxy terminus, N-term; amino terminus, IDE; immunodominant epitope, X-IDE; point mutation within IDE (where X is single letter amino acid abbreviation), EBNA1 Akata amino acid positions 367, 374, and 395 correspond to EBNA1 B95-8 amino acid positions 411, 418, and 439, respectively, and are referred to by their B95-8 amino acid positions in the literature.

Preparation of whole cell protein lysates: Briefly, HEK293 cells (CRL-1573, ATCC) were grown in DMEM supplemented with 10% FBS. HEK239 cell transfections were performed using X-tremeGENE 9 DNA Transfection Reagent (Roche). Whole cell lysates were prepared in radioimmunoprecipitation assay (RIPA) lysis buffer supplemented with 1 mM phenylmethylsulfonyl, 2 mM activated sodium orthovanadate, a 1:100 dilution of protease inhibitor and phosphatase inhibitor cocktails (Sigma-Aldrich), clarified by centrifugation, aliquoted, and stored at −80° C.

Preparation and loading EBNA1 proteins onto the slot blot: Whole cell lysates were bound to the nitrocellulose membrane according to Bio-Dot SF Microfiltration Apparatus guidelines (Bio Rad). In brief, whole cell lysates were prepared by diluting up to 10 μg of protein in 200 μL RIPA buffer to accommodate the recommended minimum loading volume. The filter papers and the nitrocellulose membrane were wetted for 10-min in 1× tris-buffered saline (TBS) before mounting onto the apparatus under vacuum flow to create an airtight seal. The membrane was first rewetted with 100 μL TBS, then loaded with 200 μL prepared protein lysate, and finally washed with 200 μL 1×TBS. The membrane was removed from the apparatus, dried for 90-min at room temperature, and stored at 4° C. for up to 1-week before antibody incubation.

Slot blot primary and secondary antibody incubation: Briefly, serum was pre-absorbed prior to primary incubation for 24-h at 4° C. in 293 cells that were prepared by fixation in 4% paraformaldehyde and permeabilization in 0.1% Triton X-100. The nitrocellulose membrane was rehydrated for 10-min in 1×TBS, then blocked in 5% non-fat dry milk in 1×TBS and incubated overnight at 4° C. with a 1:500 dilution of pre-adsorbed human sera and 1 μg/ml anti-FLAG M2 clone primary antibody (AB_259529). Membranes were washed in 1×TBS (3×5-min) followed by 1-hour incubation with fluorescently tagged secondary antibodies at room temperature in the dark, diluted in blocking buffer (3.75 μg/ml Cy3-AffiniPure goat anti-hIgA_{H} (RRID: AB_2337721); 0.03 μg/ml AlexaFluor 680-AffiniPure goat anti-hIgG_{H} (RRID: AB_2889013), 0.1 μg/ml IR800CW goat anti-mIgG_{H+L} (RRID: AB_621842). Each membrane was incubated with an optimized mixture of described secondary antibodies anti-hIgA_{H} and anti-hIgG_{H}; or anti-mIgG_{H+L} to ensure no cross reactivity between secondary antibody binding or channel fluorescence.

Slot blot analysis: The membranes were scanned on a Bio Rad ChemiDoc MP scanner, and the levels of detection in three channels were quantified using ImageLab software (Bio Rad). A result was determined to be positive when (1) a fluorescent signal in the FLAG channel appeared above background level of untransfected 293 cells and (2) a fluorescent signal in the IgA or IgG channel appeared above background level. The “normalized ratio” of values detected in the 700 nm (IgG) and 500 nm (IgA) channels (representing EBV protein detection by human sera) divided by values in the 800 nm channel (representing EBV protein levels by FLAG), were calculated. Personnel running and scoring the immunoblots were blinded to the identity and case/control status of sera at the time of analysis.

Immunoblot comparison to commercial immunoassay recomLine EBV IgG [IgA] (see, Paramita, D. K., et al. (2008). Evaluation of commercial EBV RecombLine assay for diagnosis of nasopharyngeal carcinoma. Journal of Clinical Virology, 42(4), 343-352): The commercial EBV strip-immunoassay that most closely resembles the slot blot immunoassay described here was used for comparison of methods (recomLine EBV IgG [IgA], Mikrogen). This diagnostic test uses bacterial produced, purified EBNA1 protein derived from EBV strain B95-8 sprayed onto a nitrocellulose membrane. In contrast, the slot blot uses mammalian produced EBNA1 in whole cell lysates derived from EBV strain Akata applied a nitrocellulose membrane by vacuum microfiltration. All 79 pre-diagnostic NPC sera and 79 matched controls from the SCHS and SCS were tested using the commercial kit following product guidelines. In brief, test strips were incubated with serum for 1-hour at room temperature, washed, and then incubated with peroxidase conjugated anti-human IgA antibodies for 45-min. Strips were washed and underwent a colorimetric reaction to stain sites occupied by antibodies. To evaluate, EBNA1 band intensity was compared to a “cut-off” band on each strip. EBNA1 bands below the intensity of the cut-off were considered EBNA1 negative (FIG. 8). FIG. 8 provides a summary comparison of EBNA1 IgA test results from pre-diagnostic case-control serum. The 79 pre-diagnostic NPC serum were individually matched to 79 healthy controls and grouped by years to NPC diagnosis (dx). Antibodies (IgA) against EBNA1 were detected by immunoblot, slot blot, or the commercial product recomLine EBV IgG [IgA] (Mikrogen).

The following references use spot immunoassays for antibody analysis: Herbrink P, Van Bussel FJ, Warnaar SO. The antigen spot test (AST): a highly sensitive assay for the detection of antibodies. J Immunol Methods. 1982; 48(3):293-8. doi: 10.1016/0022-1759(82)90330-1. PMID: 6174636 and Richman DD, Cleveland PH, Oxman MN. A rapid enzyme immunofiltration technique using monoclonal antibodies to serotype herpes simplex virus. J Med Virol. 1982; 9(4):299-305. doi: 10.1002/jmv.1890090408. PMID: 6286864.

### Example 3

### Test Strip

A blot may be prepared similar to the Mikrogen recomLine EBV IgG [Avidity] [IgA]

product (see, e.g., www.mikrogen.de/english/products/product-overview/testsystem/ebv-igg-aviditaet-iga.html). The various antigens may be sprayed or printed onto a nitrocellulose membrane, with deposits of the following in detectable amounts at individually-addressable locations on the membrane: the dGGA-EBNA1 protein according to any embodiment described herein, such as EBNA1dGGA_Ak or EBNA1_Library_Ak as the cancer-specific probe; a reaction control (purified human IgA); a conjugate control (HRP-conjugated secondary antibody); a cut-off control (cell lysates not expressing EBNA1); and any derivative EBNA1 protein that measures more than one EBNA1 variants (cancer-specific and non-cancer specific species including but not limited to Akata, B95-8 sequences), in any order or orientation.

In use the test strip is incubated with a patient's serum or plasma, typically diluted, incubated with peroxidase-conjugated anti human IgA antibodies, and then a peroxidase substrate is added for visualization, such as a colorimetric or chemiluminescent substrate such that a colorimetric or chemiluminescent reaction at the location of the dGGA-EBNA1 protein is indicative of a positive reaction. Alternatively, the proteins can be loaded onto individually-addressable beads for flow cytometry. In either case, one or more steps, or the entire reaction may be automated, including detection, and analysis of the strip or beads may be performed in a computer, using a computer-implemented method.

It should be recognized that the choice of control proteins for the above-described blots or beads may be varied, added to, or limited by one of ordinary skill in the art, so long as the assay produces valid results. For example, one or more, or all of the additional EBNA1 protein derivatives may be omitted.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A device comprising:
a protein-binding substrate; and
bound to the substrate, an isolated EBV EBNA1 protein produced in a mammalian cell that binds serum IgA of an NPC patient, the protein comprising an EBV EBNA1 protein with at least 50% of the GA region deleted.

2. The device of claim 1, wherein the EBV EBNA1 protein comprises a sequence of SEQ ID NO: 1 in which at least 120 contiguous amino acids between amino acids 89 and 328 are deleted.

3. The device of claim 1, wherein the EBV EBNA1 protein comprises a sequence of SEQ ID NO: 2 in which at least 95 contiguous amino acids between amino acids 91 and 284 are deleted.

4. The device of claim 1, wherein the EBV EBNA1 protein comprises a sequence of SEQ ID NO: 1 or SEQ ID NO: 2 in which at least 75% or at least 90% of the GA region is deleted.

5. (canceled)

6. The device of claim 1, wherein the EBV EBNA1 protein comprises a sequence of SEQ ID NO: 1 or SEQ ID NO: 2 in which the GA region is deleted.

7. The device of claim 1, wherein the EBV EBNA1 protein comprising a sequence of SEQ ID NO: 1, comprises one, two, or three of the amino acid substitutions E411D, H418L, and A439T.

8. The device of claim 1, wherein the EBV EBNA1 protein comprises at least an immunodominant epitope of SEQ ID NO: 2.

9. The device of claim 1, wherein the EBV EBNA1 protein has a higher amino acid sequence identity to an EBNA1 present in a nasopharyngeal carcinoma than SEQ ID NO: 1.

10. The device of claim 1, wherein the EBV EBNA1 protein comprises an amino acid sequence having at least 95% sequence identity with SEQ ID NO: 2.

11. The device of claim 1, wherein the modified EBV EBNA1 protein has the amino acid sequence of SEQ ID NO: 4.

12. The device of claim 1, wherein the substrate is a wettable membrane or a bead.

13. (canceled)

14. The device of claim 1, wherein the substrate is a wettable membrane and the modified EBV EBNA1 protein is bound to the membrane at two or more discrete locations, such as a slot blot or a dot blot.

15. The device of claim 14, further comprising positive and negative control proteins deposited at one or more discrete locations on the membrane.

16. The device of claim 15, comprising one or more positive control proteins deposited at one or more discrete locations on the membrane, wherein the positive control protein is human IgA.

17. A kit, comprising a device of claim 1, contained within packaging.

18. The kit of claim 17, further comprising anti-human-IgA primary antibody, optionally contained within a vessel.

19. (canceled)

20. The kit of claim 17, further comprising a labeled secondary antibody that binds specifically to the anti-human IgA primary antibody.

21-22. (canceled)

23. The kit of claim 17, wherein the substrate is a bead for use in flow cytometry, and the label is fluorescent.

24-27. (canceled)

28. A method of treating a patient, comprising:
monitoring the patient for risk of developing NPC by determining if the patient has serum anti-EBV EBNA1 IgA antibodies by determining if serum IgA antibodies of the patient bind to an EBV EBNA1 protein produced in a mammalian cell that binds serum IgA of an NPC patient, the protein comprising an EBV EBNA1 protein with at least 50% of the GA region deleted using a device of claim 1, and
when the patient has serum IgA antibodies that bind to the EBV EBNA1 protein, monitoring the patient for development of NPC, and when present, treating the patient for NPC.

29-33. (canceled)

34. A method of determining risk of development of NPC in a patient, comprising: determining if the patient has serum anti-EBV EBNA1 IgA antibodies by determining if serum IgA antibodies of the patient bind to an EBV EBNA1 protein produced in a mammalian cell that binds serum IgA of an NPC patient, the protein comprising an EBV EBNA1 protein with at least 50% of the GA region deleted using a device of claim 1.


SEQUENCE LISTING (5 sequences)

SEQ ID NO: 1 - protein, 641 aa, synthetic construct
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 2 - protein, 597 aa, synthetic construct
MSDEGPGTGPGNGLGQKEDSSGPEGSGGSGPQRRGGDNHGRGRGRGRGRGGGRPGAPGGSGSGPRHRDGVRRPQKRPSCIGCKGAHGGTGTGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGGAGAGGAGAGGAGGAGAGGAGAGGAGGAGAGGAGAGGAGGAGAGGAGGAGAGGAGGAGGGGAGGAGGGGRGRGGSGGRGRGGSGGRGRGGSGGRRGRGRERARGRSRERARGRGRGRGEKRPRSPSSQSSSSGSPPRRPPPGRRPFFHPVGDADYFEYLQEGGPDGEPDVPPGAIEQGPTDDPGEGPSTGPRGQGDGGRRKKGGWFGKHRGQGGSNPKFENIAEGLRVLLARSHVERTTEEGNWVAGVFVYGGSKTSLYNLRRGIALAVPQCRITPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 3 - protein, 405 aa, synthetic construct
MSDEGPGTGPGNGLGQKEDSSGPEGSGGSGPQRRGGDNHGRGRGRGRGRGGGRPGAPGGSGSGPRHRDGVRRPQKRPSCIGCKGAHGGTGTGRGRGGSGGRGRGGSGGRGRGGSGGRRGRGRERARGRSRERARGRGRGRGEKRPRSPSSQSSSSGSPPRRPPPGRRPFFHPVGDADYFEYLQEGGPDGEPDVPPGAIEQGPTDDPGEGPSTGPRGQGDGGRRKKGGWFGKHRGQGGSNPKFENIAEGLRVLLARSHVERTTEEGNWVAGVFVYGGSKTSLYNLRRGIALAVPQCRITPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 4 - protein, 419 aa, synthetic construct
MSDEGPGTGPGNGLGQKEDSSGPEGSGGSGPQRRGGDNHGRGRGRGRGRGGGRPGAPGGSGSGPRHRDGVRRPQKRPSCIGCKGAHGGTGTGGAGGAGAGGAGGGGRGRGGSGGRGRGGSGGRGRGGSGGRRGRGRERARGRSRERARGRGRGRGEKRPRSPSSQSSSSGSPPRRPPPGRRPFFHPVGDADYFEYLQEGGPDGEPDVPPGAIEQGPTDDPGEGPSTGPRGQGDGGRRKKGGWFGKHRGQGGSNPKFENIAEGLRVLLARSHVERTTEEGNWVAGVFVYGGSKTSLYNLRRGIALAVPQCRITPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 5 - DNA, 111 bp, synthetic construct
  source 1..111: mol_type = other DNA
aattcgcgatcgcttaattaaaccatggactacaaagaccatgacggtgattataaagatcatgacatcgattacaaggatgacgatgacaagcttgcggccgcggtaccg